(19) United States
(12) Patent Application Publication
(10) Pub. No.: US 2026/0265327 A1
(43) Pub. Date: Sep. 10, 2026

(54) Anktiva and Lymphocyte Rescue Compositions and Methods

(71) Applicant: ImmunityBio, Inc., San Diego, CA (US)

(72) Inventors: Patrick Soon-Shiong, Culver City, CA (US); Adrian Rice, Culver City, CA (US)

(73) Assignee: ImmunityBio, Inc., San Diego, CA (US)

(21) Appl. No.: 19/557,689

(22) Filed: Mar. 5, 2026

Related U.S. Application Data
(60) Provisional application No. 63/768,448, filed on Mar. 7, 2025.
(60) Provisional application No. 63/769,440, filed on Mar. 10, 2025.
(60) Provisional application No. 63/800,326, filed on May 5, 2025.
(60) Provisional application No. 63/813,140, filed on May 28, 2025.
(60) Provisional application No. 63/832,845, filed on Jun. 30, 2025.
(60) Provisional application No. 63/882,096, filed on Sep. 15, 2025.

(51) Int. Cl. C07K 14/54; A61K 38/00; A61K 45/06; A61P 35/00

(52) U.S. Cl. CPC C07K 14/5443; A61K 45/06; A61P 35/00; A61K 38/00; C07K 2319/30

(57) ABSTRACT

A method for treating lymphopenia and cancer comprises administering an IL-15N72D:IL-15RαSu/Fc complex (N-803) containing a dimeric IL-15RαSu/Fc and two IL-15N72D molecules, and further administration of at least one further cancer therapy. This approach aims to restore immune function in patients who have undergone cancer therapies causing lymphopenia, including chemotherapy, radiotherapy, or immunotherapy with checkpoint inhibitors. As such, even failed treatments can be continued. The method may also include administering CAR NK cells to enhance the immune response against cancer. In many cases, such treatment significantly increases median overall survival.

This application claims the benefit of copending US Provisional Applications with the serial numbers 63/882,096, filed Sep. 15, 2025, 63/832,845, filed Jun. 30, 2025, 63/813,140, filed May 28, 2025, 63/800,326, filed May 5, 2025, 63/769,440, filed Mar. 10, 2025, and 63/768,448, filed Mar. 7, 2025, each of which are incorporated by reference herein.

### FIELD OF THE INVENTION

The field of the invention is composition and methods to treat lymphopenia in an individual undergoing cancer treatment, and especially where the treatment is chemotherapy, radiation therapy, immune checkpoint inhibition therapy, and/or cancer vaccine therapy and where the treatment is associated with lymphopenia.

### SEQUENCE LISTING

The content of the XML file of the sequence listing named 102690.0079.xml, which is 5,159 bytes in size, created on Feb. 5, 2026 and electronically submitted via Patent Center along with the present application, is incorporated by reference in its entirety.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Lymphopenia (also known as lymphocytopenia) is often associated with increased cancer risk, higher susceptibility to various infections, and patient survival in chemotherapy.

While lymphopenia may not have a single cause (idiopathic lymphopenia), lymphopenia is frequently caused by various cancer treatments and particularly chemotherapy and radiation therapy. As a result, such treated cancer patients typically have a compromised immune system, which runs counter to the goal of cancer eradication. To address lymphopenia, several attempts have been undertaken. For example, IL-2, IL-7, and IL-15 were evaluated as these cytokines are known to play a role in the development, proliferation, and survival of T cells (see e.g., Journal for Immuno Therapy of Cancer (2019) 7:85). Unfortunately, IL-2 and IL-15 are known to have significant toxicity and to stimulate Treg cells, rendering such treatment less effective. To circumvent toxicity and Treg stimulation, ALT-803 has been proposed as a rescue agent to increase lymphocyte count after cancer treatment as is described in US 2021/0188933. While inducing NK cell proliferation, and to a lesser degree proliferation of CD8+ and CD4+ T cells, in healthy volunteers, the efficacy for lymphocyte rescue in subjects with lymphopenia, and in particular in cancer patients with treatment-induced lymphopenia and concurrent treatment failure was not demonstrated, especially in the context of increased survival.

Thus, even though various compositions and methods of addressing lymphopenia are known in the art, all or almost all of them suffer from several drawbacks. Therefore, there remains a need for improved systems and methods for cancer treatments that preserve or improve lymphocyte count.

### SUMMARY OF THE INVENTION

The inventive subject matter is directed to compositions and methods of treatment for subjects with lymphopenia, and particularly subjects suffering from cancer in which prior treatment has led to lymphopenia. In at least some embodiments, the subjects had previously undergone one or more lines of treatment, and presently experience or are likely to experience treatment failure of a current treatment (e.g., checkpoint inhibitor and/or chemotherapeutic agent). Advantageously, contemplated compositions and methods are demonstrated to reverse, or at least partially reverse lymphopenia and significantly increase overall survival.

In one aspect of the inventive subject matter, the inventors contemplate a method of treatment of a cancer patient with lymphopenia, wherein the lymphopenia was induced by prior cancer treatment. Such method typically includes a step of administering to the patient an effective amount of an IL-15N72D:IL-15RαSu/Fc complex (N-803) formulated for administration to a subject, wherein said N-803 comprises a dimeric IL-15RαSu/Fc and two IL-15N72D molecules. Most typically, the N-803 is administered over a period and at a schedule effective to increase absolute lymphocyte count (ALC) to thereby increase median overall survival.

For example, contemplated prior cancer treatments include checkpoint inhibitor treatment, radiation treatment, chemotherapy, and/or immune therapy. Moreover, it is contemplated that the N-803 is administered subcutaneously over at least four dosages, and/or that the ALC is increased to at least 1,000 cells/μL. Where suitable, contemplated methods will also include a step of co-administering at least one of a checkpoint inhibitor, a cancer vaccine, a cell-based product, and granulocyte colony stimulating factor (G-CSF) and a cytokine. For example, contemplated cell-based products include a memory like cytokine enhanced NK (m-ceNK) cell, a cytokine induced memory-like (CIML) NK cell, an autologous NK cell, an NK cell expressing a high-affinity CD16 receptor (haNK cell), or a targeted haNK cell expressing a chimeric antigen receptor that binds to a tumor epitope. In further embodiments, the cancer patient has a solid tumor.

In another aspect of the inventive subject matter, the inventors contemplate a method of increasing median overall survival in a cancer patient with lymphopenia, wherein the lymphopenia was induced by prior cancer treatment. Such method will typically include a step of co-administering to the patient (a) a cancer treatment and (b) an effective amount of an IL-15N72D:IL-15RαSu/Fc complex (N-803) formulated for administration to a subject, wherein said N-803 comprises a dimeric IL-15RαSu/Fc and two IL-15N72D molecules.

While not always the case, the cancer treatment may be the prior cancer treatment. Alternatively, it is contemplated that the cancer treatment comprises administration of at least one of a checkpoint inhibitor, a cancer vaccine, a cell-based product, and granulocyte colony stimulating factor (G-CSF) and a cytokine. It is further contemplated that the effective amount will be effective to increase the lymphocyte count of the patient to an ALC that is at least 1,000 cells/μL. Preferably, the median overall survival is at least 4 months, and most typically the cancer patient has a solid tumor (such as non-small-cell lung cancer (NSCLC), small-cell lung cancer (SCLC), urothelial cancer, head and neck squamous cell cancer, melanoma, renal cancer, gastric cancer, cervical cancer, hepatocellular carcinoma (HCC), colorectal cancer, or metastatic pancreatic cancer).

In a still further aspect of the inventive subject matter, the inventors contemplate a method of increasing median overall survival in a relapsed cancer patient with lymphopenia, wherein the lymphopenia was induced by prior cancer treatment. Such method will typically include a step of administering to the patient an effective amount of an IL-15N72D:IL-15RαSu/Fc complex (N-803) formulated for administration to a subject, wherein said N-803 comprises a dimeric IL-15RαSu/Fc and two IL-15N72D molecules, and a further step of continuing the prior treatment while the patient receives the N-803.

For example, in at least some embodiments, the patient has relapsed on checkpoint inhibitor treatment (e.g., targeting PD-1 or CTLA-4). Where desired, such methods may further include a step of co-administering a cancer vaccine and/or a cell-based therapy. For example, suitable cancer vaccines may comprise a recombinant adenovirus that comprises a nucleic acid that encodes at least one of CEA, MUC-1, and Brachyury, or a tumor and patient specific neoepitope, while suitable cell-based therapies may comprise a memory like cytokine enhanced NK (m-ceNK) cell, a cytokine induced memory-like (CIML) NK cell, an autologous NK cell, an NK cell expressing a high-affinity CD16 receptor (haNK cell), or a targeted haNK cell expressing a chimeric antigen receptor that binds to a tumor epitope.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 is a table depicting exemplary markers followed in a human study.

FIG. 2 depicts exemplary results for selected markers and immune signaling pathways at 12 weeks comparing TriAd5 treatment with TriAd5+N-803 treatment.

FIG. 3 depicts exemplary results for selected T cell NK cell markers and immunosuppressive analytes after 12 weeks of treatment with TriAd5+N-803 treatment versus baseline.

FIG. 4 depicts exemplary results for immune function related markers at 12 weeks comparing TriAd5 treatment with TriAd5+N-803 treatment.

FIG. 5 is an exemplary schematic depicting an experimental setup to determine efficacy and synergy of co-administration of N-803 and a cancer vaccine in subjects with lymphopenia.

FIG. 6 is an exemplary schematic depicting mean ALC values increase over time with lymphopenia reversal ≥1,000 (N=265) in cancer patients.

FIG. 7 is an exemplary schematic depicting mean percentage change from baseline in ALC values over time by baseline median ALC (N=265) in cancer patients.

FIG. 8 is an exemplary schematic depicting maintenance of ALC count (ALC≥1,500) over time by N-803 as measured by ALC (N=265) in cancer patients.

FIG. 9 is an exemplary schematic depicting maintenance of ALC≥1,500 across all tumor types as measured by percentage change of ALC (N=265) in cancer patients.

FIG. 10 depicts significant prolongation of overall survival in 3^{rd} to 6^{th} line metastatic pancreatic cancer patients with reconstitution of lymphocytes by in-vivo and ex-vivo lymphocyte stimulating agents independent of tumor burden stratified by ALC levels.

FIG. 11 depicts significant prolongation of overall survival in 3^{rd} to 6^{th} line metastatic pancreatic cancer patients with reconstitution of lymphocytes by in-vivo and ex-vivo lymphocyte stimulating agents with lower tumor burden stratified by NLR ratio and tumor burden.

FIGS. 12A and 12B depicts exemplary effects of ALC levels (≥1,000) on overall survival: subjects with prolonged survival achieve an ALC≥1,000 with lymphocyte rescue (12A). The long-tail in the Kaplan Meyer plot (12B) of 7.9 Months (95% CI: 6.8, 10.3) is seen with subjects who achieve an ALC≥1,000 and maintain treatment beyond cycle 5 independent of tumor burden in 3^{rd} to 6^{th} line metastatic pancreatic cancer.

FIGS. 13A and 13B depicts an exemplary effect of tumor burden and ALC levels on overall survival: Subjects with lower tumor burden and lymphocyte rescue (ALC≥1,000) (13A) demonstrate a prolonged survival of 10.1 months (95% CI: 6.8, 15.2) (13B) compared to subjects with higher tumor burden (CA19-9 Median≥3,852 u/mL) and in severe lymphopenia (ALC<1,000).

FIG. 14 depicts exemplary nogapendekin alfa inbakicept (N-803; ANKTIVA).

FIG. 15 depicts exemplary mOS for CA19-9≤ or >3,852 U/mL (baseline median) in the upper panel. mOS for participants who survived ≥5 months (median survival time) by CA19-9 level was < or ≥1,516 U/mL (median baseline level for these participants) is shown in the middle panel, and mOS for NLR≤ or >4.27 (baseline median) is shown in the bottom panel.

FIG. 16 depicts exemplary study design for Tri-Ad/Yeast+N-803 Lymphopenia Study in C57BL/6 Mice.

FIG. 17 depicts exemplary absolute lymphocyte count data for Lynch Syndrome.

FIG. 18 depicts exemplary absolute lymphocyte count data for castration resistant prostate cancer.

FIG. 19 depicts a Kaplan Meier plot of mOS stratified by degree of lymphopenia.

FIG. 20 depicts a Kaplan Meier plot for various cohorts in human subjects.

FIG. 21 depicts a Kaplan Meier plot of mOS stratified by degree of lymphopenia in subjects with NSCLC.

FIG. 22 depicts results for sustained lymphocyte counts in two cohorts characterized by mild or severe lymphopenia.

FIG. 23 depicts a Kaplan Meyer plot for subjects with sustained ALC.

FIG. 24 depicts a Kaplan Meyer plot for subjects with sustained ALC considering tumor burden.

FIG. 25 depicts lymphocyte counts in two cohorts characterized by mild or severe lymphopenia.

FIG. 26 depicts exemplary serum markers pre- and post N-803 infusion.

FIG. 27 depicts results for various cell types of immune relevant cells pre- and post N-803 infusion.

FIG. 28 depicts results for various cell types of monocytes pre- and post N-803 infusion.

FIG. 29 depicts results for various markers for immune relevant cells pre- and post N-803 infusion.

FIG. 30 depicts a tSNE graph for various immune relevant cells pre- and post N-803 infusion.

### DETAILED DESCRIPTION

The inventors have unexpectedly discovered various therapeutic compositions and methods of lymphocyte rescue in which a subject with lymphopenia and cancer can be treated to concurrently increase lymphocyte count and restore or improve efficacy of cancer immune therapy. In at least some embodiments, the pharmaceutical components of such treatment will exert a synergistic effect with respect to lymphocyte rescue and/or efficacy of immune therapy (e.g., cancer vaccine therapy). Significantly, the inventors also discovered that the treatments presented herein can significantly increase overall survival in a variety of cancers, even in the presence of treatments that would otherwise lead to or exacerbate lymphopenia.

As will be appreciated, contemplated prior cancer treatments include all currently known treatments that result or aggravate lymphopenia and as such include treatments with one or more alkylating agents, topoisomerase inhibitors, nucleoside analogs, DNA repair inhibitors, kinase inhibitors, chemotherapeutic agents, anti-metabolites, hormones, checkpoint inhibitors, antibodies that interfere with cell signaling, gamma radiation therapy, and radiation therapy with implanted alpha emitters, etc., which are most typically administered over extended periods typically between a minimum effective dosage (e.g., metronomically) and the maximum tolerated dosage. Therefore, contemplated causes and/or severity of lymphopenia may vary considerably. Additionally, it is contemplated that the individual with lymphopenia may have been diagnosed with an idiopathic lymphopenia or lymphopenia due to a viral infection (e.g., HIV virus).

Thus, lymphocyte counts at the start of (co-)administration of N-803 in the individual may be between 100-200, or between 200-400, or between 250-500, or between 500-700, or between 600 and 900, or between 750-999. Thus, lymphocyte counts of less than 900, or less than 800, or less than 700, or less than 600, or less than 500, or less than 400, or less than 300, but more than 50 or more than 100 or more than 150 or more than 200 are especially contemplated. In general, and unless noted otherwise herein, lymphopenia is characterized by a low absolute lymphocyte count (ALC)<1,000 cells/L and in children by an ALC<3,000 cells/μL.

Unfortunately, treatment-induced lymphopenia due to radiation, chemotherapy or immunotherapy, is often associated with significantly lower overall survival across multiple tumor types. To the best of the inventors' knowledge, no treatments were available to address lymphopenia in cancer therapy with the goal of maintaining ALC levels >1,000 cells/L that will ultimately support prolonging overall survival. The inventors have now discovered that patients with lymphopenia and current treatment failure (e.g., CPI, chemotherapy, cancer vaccine therapy, etc.) can in fact continue with the same treatment as N-803 rescues and maintains ALC at or above certain cell counts, such as >1,000 cell/μL. Most notably, N-803 as is shown in more detail below, the so achieved lymphocyte rescue will extend overall survival.

As used herein, the terms “N-803”, “NAI”, “Nogapendekin Alfa Inbakicept”, and ANKTIVA™ (Nogapendekin Alfa Inbakicept, commercially available from Immunitybio, Inc.) are used interchangeably and refer to an IL-15N72D:IL-15RαSu/Fc complex formulated for administration to a subject in which the N-803 complex may include a dimeric IL-15RαSu/Fc portion and two IL-15N72D molecules. U.S. Pat. No. 9,328,159, which describes N-803, is incorporated herein by reference in its entirety. However, it should be appreciated that various mutant forms and variations of N-803 are also deemed suitable for use herein, alone or in combination with N-803. For example, the IL-15N72D component may be a mutant form of human IL-15 with an asparagine to aspartic acid substitution at position 72. This mutation may enhance the biological activity of IL-15. The IL-15RαSu component may be a soluble form of the IL-15 receptor alpha chain. The Fc portion may be derived from human IgGI. Most typically, the N-803 complex may have a molecular weight of approximately 114 kDa and will advantageously exhibit an extended serum half-life compared to native IL-15 due to the Fc fusion. Like IL-15, IL-15:IL-15Rα and fusion constructs derived therefrom, N-803 has specific agonist activity at cell surface-expressed IL-15Rbg. The inventors therefore conceive of the inventive anti-lymphopenia properties of N-803 as extending to all stabilized IL-15 and IL-15:IL-15Rα fusion constructs.

As discussed in more detail below, and among further benefits, it should also be noted that N-803 may stimulate proliferation and activation of NK cells and memory CD8+ T cells and monocytes without stimulating Treg cells. Moreover, N-803 will also induce production of cytokines like IFN-γ. Therefore, N-803 and variants thereof may enhance antigen-specific T cell responses when combined with vaccines. Suitable variants of N-803 may include complexes with different IL-15 mutations, such as IL-15N72A. The Fc portion may be derived from other IgG subtypes. Linker sequences between the components may be modified. The ratio of IL-15 to IL-15Rα may be altered. Exemplary sequences for suitable N-803 and analogs are shown in SEQ ID NO:1-4, which are part of the sequence listing entitled “102690.0079PRO.xml” having a file size of 6 KB, created Mar. 3, 2025, which is incorporated by reference and which forms express part of this disclosure.

For example, in one aspect of the inventive subject matter, it is contemplated that subcutaneous co-administration of N-803 with an adenovirus-based cancer vaccine may provide in a subject previously diagnosed with lymphopenia enhanced lymphocyte rescue while also enhancing anti-cancer effect of the vaccine component. Most typically, lymphopenia in the subject was caused by prior anti-cancer treatment, such as by radiation therapy and/or chemotherapy.

Therefore, in some exemplary embodiments, a treatment method may comprise administering to a patient an effective amount of 1) an adenoviral vector comprising at least one target antigen, and 2) an IL-15N72D:IL-15RαSu/Fc complex (N-803) formulated for administration to a subject. Most typically, the patient was previously diagnosed with lymphopenia, which may have been due to a prior cancer treatment (or which may have been due to a viral infection or an idiopathic lymphopenia) and is currently diagnosed with a cancer. In addition, it is typically preferred that the N-803 may comprise a dimeric IL-15RαSu/Fc and two IL-15N72D molecules.

In some examples, the adenoviral vector may comprise a replication defective adenoviral vector subtype 5 (Ad5) comprising a deletion in the E1 region and a deletion in the E2b region. In some embodiments, the Ad5 vector may comprise a nucleic acid encoding CEA, Brachyury, and/or MUC-1. In certain embodiments, the Ad5 vector may comprise a Tri-Ad5 vaccine, wherein the Tri-Ad5 vaccine may comprise an Ad5 vector comprising a nucleic acid encoding CEA, an Ad5 vector comprising a nucleic acid encoding Brachyury, and an Ad5 vector comprising a nucleic acid encoding MUC-1. However, it should be appreciated that the Ad5 vector may also include a nucleic acid that encodes one or more cancer specific antigens (e.g., PSA, PSMA, etc.) or one or more tumor and patient specific neoepitopes. As will be readily appreciated, where multiple antigens are encoded in the nucleic acid, the antigen sequence portions may be arranged in a minigene, a polytope, or in multiple cistronic arrangements. Furthermore, contemplated Ad5 particles may further include a nucleic acid portion that encodes for one or more co-stimulatory factors, cytokines, and/or checkpoint inhibitors.

In still further contemplated aspects, it should be appreciated that the cancer vaccine need not necessarily be limited to an Ad5-based vaccine, but that numerous alternative therapeutic viruses are also deemed suitable for use herein, including various adeno-associated viruses (AAV), retroviruses, lentiviruses, herpes simplex viruses (HSV), alphaviruses, and poxviruses. Similarly, alternative cancer vaccines also include recombinant yeast cells and even recombinant bacterial cells (e.g., ClearColi™ from Research Corporation Technologies) so long as such bacterial cells express at least one antigen relevant to the treatment of cancer.

Where the vaccine is a recombinant virus, it is generally contemplated that the recombinant antigen is expressed such that the expressed antigen is directed to the cytoplasm (e.g., for preferential MHC-I presentation), and/or that the expressed antigen is directed to the endosomal or lysosomal compartment (e.g., for preferential MHC-II presentation).

Alternatively, or additionally, the cancer vaccine may be a yeast-based vaccine, for example, as disclosed in the publication WO2019222073A1 (filed May 15, 2018, published Nov. 21, 2019, Globeimmune Inc.), which is incorporated by reference herein in its entirety. As further described therein, yeast lysates may be engineered to present tumor-associated antigens, which are then used to stimulate antigen-specific T-cell responses, particularly cytotoxic CD8+ T cells, in subjects. Yeast may be wild type or may be genetically engineered to comprise a nucleic acid encoding a tumor antigen or a neoepitope. Yeast may be formulated for subcutaneous, intravenous, or intratumoral administration.

In most embodiments, it is contemplated that the co-administration of N-803 and the cancer vaccine will be contemporaneous and may be performed in a single dosage unit containing the N-803 and the cancer vaccine, or in two separately administered dosages. In this context, contemporaneous should be understood to mean administration of both components within equal or less than 24 hours, or more typically equal or less than 12 hours, equal or less than 8 hours, equal or less than 6 hours, equal or less than 4 hours, equal or less than 2 hours, or equal or less than 1 hour, and even less. With respect to the co-administration, it is further contemplated that the N-803 and the cancer vaccine are administered to the same site or compartment, and in most typical scenarios administration will be subcutaneous. However, it should be noted that the N-803 may be administered to a first site (e.g., subcutaneously) while the vaccine is administered to a different, second site (e.g., intravenously, intratumorally, etc.). Alternatively, both the antigenic sequence(s) and N-803 may be encoded by a viral vector and formulated for intravenous administration.

As will be readily appreciated, N-803 may be administered to patients with lymphopenia in various dosages, schedules, and routes of administration, most typically depending on the severity of lymphopenia. Therefore, contemplated dosages of N-803 may range from about 1 μg/kg to about 100 μg/kg. In some embodiments, the dosage may be about g/kg, 15 μg/kg, 20 μg/kg, or 25 μg/kg. Likewise, the administration schedule for N-803 may vary depending on the patient's condition and response. N-803 may be administered once weekly, once every two weeks, or once every three weeks. In certain embodiments, N-803 may be administered in cycles, such as 3 weekly doses followed by 3 weeks off. The treatment course may continue for 4-12 cycles or more. As noted above, N-803 may be administered through various routes, including subcutaneous, intravenous, intramuscular, or intradermal injection. Subcutaneous administration may be preferred in some cases for ease of use and sustained release.

Following N-803 administration, patients may experience increases in lymphocyte counts. The absolute lymphocyte count may increase by about 1.5-fold to 5-fold compared to baseline levels. This increase may be observed within 1-2 weeks after the first dose and may peak around 2-4 weeks. Specific lymphocyte subsets that may increase include NK cells, CD8+ T cells, and memory T cells. NK cell counts may increase by about 3-fold to 10-fold. CD8+ T cell counts may increase by about 2-fold to 8-fold. Memory T cell populations may expand by about 1.5-fold to 6-fold. Of course, it should be recognized that the magnitude and duration of lymphocyte increases may vary between patients. Some patients may maintain elevated lymphocyte levels for several weeks after discontinuing N-803 treatment. Repeated dosing of N-803 may lead to more sustained increases in lymphocyte counts over time.

With respect to the administration of the cancer vaccine component it is generally contemplated that the particular type of cancer vaccine component will at least in part determine the quantity administered. However, it is generally contemplated that where the cancer vaccine component is a recombinant virus, administration will typically be in the range of 1-10×10⁸ viral particles, and more typically in the range of 1-10×10⁹ viral particles, and most typically in the range of 1-10×10¹⁰ viral particles, and in some cases even higher. Among other suitable recombinant viruses, especially contemplated recombinant viruses include Ad5-based viruses with a deletion of E1 and E2b genes, and exemplary viral constructs and methods are described in U.S. Pat. No. 11,779,637, incorporated by reference in its entirety herein. On the other hand, where the cancer vaccine component is a recombinant yeast, administration will typically be in the range of 1-10 yeast units, and more typically in the range of 5-50 yeast units, and most typically in the range of 10-100 yeast units, and in some cases even higher, and with a yeast unit being 1×10⁷ yeast particles per unit. For example, especially contemplated yeast vaccines will include recombinant Saccharomyces, Kluyveromyces, Pichia, Hansenula cells and lysates thereof. Most typically, the lysates are further processed to remove at least some of the membrane and cell wall fraction. Among other suitable compositions and methods, particularly preferred yeast preparations are described in U.S. Pat. No. 12,128,078, incorporated by reference in its entirety herein. Where the cancer vaccine component is a recombinant bacterium (e.g., ClearColi™), administration will typically be in the range of 1-10×10⁷ cfu (colony forming units), and more typically in the range of 1-10×10⁸ cfu, and most typically in the range of 1-10×10⁹ cfu, and in some cases even higher.

As will be readily appreciated, the cancer vaccine component and the N-803 will be generally formulated for injection or infusion, and the concentration will generally depend on the desired ultimate dosage and manner of administration. Therefore, it is generally preferred that the concentration and pharmaceutically acceptable carrier will be selected such as to allow administration of therapeutic quantities for subcutaneous administration, intravenous infusion, intratumoral injection, or intramuscular injection. However, in other embodiments, a step of ex vivo contacting a PBMC fraction of the subject with the cancer vaccine component and N-803 is also contemplated, followed by infusion of the mixture.

In further contemplated aspects, the co-administration of N-803 with the (adenovirus-based) cancer vaccine may result in various immunological effects. For example, dendritic cells may be activated, and granzyme and perforin may be upregulated. Interleukin-15 (IL-15) may be upregulated, while Interleukin-10 (IL-10) signaling may be decreased. Interferon gamma (IFNγ) signaling may be upregulated, and crosstalk between dendritic cells and natural killer (NK) cells may be increased. Additionally, crosstalk between dendritic cells and T cells may be increased, and Th1 signaling may be upregulated. Moreover, such combination therapy may lead to activation of various immune cell populations. NK cells may be activated, and CD4 T cells, CD8 T cells, and memory T cells may be activated. In some embodiments, T cell activation may peak at 12 weeks.

In addition to the above, it should be noted that the treatment compositions and methods presented herein are not limited to (co-)administration of N-803 and a cancer vaccine, but that the cancer vaccine may be augmented or replaced by other cancer treatments, and especially immune therapy and/or cell-based therapy such as autologous or heterologous NK cells, NK cells that have been genetically engineered to express a high affinity CD16 receptor and/or a cancer targeting chimeric antigen receptor (CAR) such as an anti-PD-L1 targeting CAR, or cytokine enhanced NK cells such as m-ceNK cells. The above and additional treatments are believed to be particularly beneficial for patients that have been previously treated with radiotherapy, chemotherapy, and/or immunotherapy. In some cases, the immunotherapy may comprise an immune checkpoint inhibitor, such as an anti-PD-1, anti-PD-L1, or anti-CTLA4 antibody. Examples of immune checkpoint inhibitors may include avelumab, nivolumab, atezolizumab, durvalumab, tremelimumab, or pembrolizumab. Therefore, in certain embodiments, the combination therapy may be used to treat patients with immune checkpoint inhibitor induced lymphopenia. Such methods may comprise administering N-803 to a patient who has been previously treated with a first immune checkpoint inhibitor. In some cases, a second immune checkpoint inhibitor may be administered in combination with N-803. The N-803 and the second immune checkpoint inhibitor may be administered simultaneously or sequentially. Therefore, it is contemplated that suitable patients include those with 2^{nd}, 3^{rd}, 4^{th}, and higher line of treatment failure with one or more cancer treatments where the patient suffers from lymphopenia.

Consequently, in still further contemplated embodiments, suitable methods may further comprise administering tumor targeting CAR Natural Killer (CAR NK) cells, m-ceNK cells, or a CAR T cells in combination with N-803. The CAR NK cell may comprise a natural killer cell line, such as an NK-92 cell, or a primary human NK cell. In certain embodiments, the primary human NK cell may be a memory like cytokine enhanced NK (m-ceNK) cell, or a cytokine induced memory-like (CIML) NK cell. The CAR may comprise an extracellular binding domain with specificity for PD-L1, CD19, EGFR, or HER2.

In certain embodiments, the patient may have previously received chemotherapy and/or radiotherapy. The method is particularly applicable to patients who have been treated with chemotherapeutic agents selected from the group consisting of taxanes, platinum-based agents, cyclophosphamide, fludarabine, mTOR inhibitors, tyrosine kinase inhibitors, or methotrexate.

These agents are known to induce lymphodepletion, and subsequent immune restoration using the disclosed cytokine combination is likely to enhance post-treatment immune surveillance and recovery.

Viewed from a different perspective and regardless of the particular therapeutic agent and prior treatment failure, it is contemplated that administration of N-803 will allow for lymphocyte rescue in an amount that is sufficient to at least partially restore the function of the patient's immune system. Adding cell-based therapies as noted above if believed to further enhance the patient's immune function, and with that overall survival. Thus, it should be appreciated that the combination of N-803 with (e.g., an adenovirus-based) a cancer vaccine or other immune therapy (e.g., checkpoint inhibitor, cell-based therapy) may provide a synergistic approach to enhancing lymphocyte rescue and promoting anti-tumor immunity.

In still further aspects, the inventors contemplate a method of treatment of lymphopenia in a subject suffering from immunosuppression due to an infectious disease. Specifically, the method includes the administration of interleukin-7 (IL-7) or a stabilized derivative thereof, in combination with an IL-15 superagonist complex, such as N-803.

In other embodiments, the method is employed in patients diagnosed with cancer, for example non-small cell lung cancer (NSCLC). Additionally, the method may be applied to patients suffering from infectious diseases characterized by immune suppression or lymphocyte dysfunction. In such cases, the combined administration of IL-7 and N-803 offers a strategy to bolster immune competence and improve pathogen clearance.

In still further contemplated aspects embodiments, N-803 or other IL-15 agonist is administered alone and is the sole therapeutic agent for the treatment of the disease, disorder, condition, or infection as described herein. However, more typically, the inventors contemplate a method for generating a protective bioshield against cancer progression through the co-administration of N-803 and at least one immunotherapy agent. The term “bioshield” as used herein, refers to a therapeutically induced state of enhanced immune competence that provides sustained immunological defense against diseases such as tumor growth and metastasis. The immunotherapy agent may be any known immune-stimulating or immunomodulatory component, including but not limited to checkpoint inhibitors (CPI) such as anti-PD-1, anti-PD-L1, or anti-CTLA-4 antibodies; NK cell therapies; memory-like cytokine-enhanced NK (m-ceNK) cell therapies; tumor-targeted antibody-NK conjugates (t-haNK); recombinant adenoviral vectors (e.g., Ad5V) encoding tumor-associated antigens; yeast-based immunotherapies; Class I histone deacetylase (HDAC) inhibitors; and cytokines such as IL-7 or IL-21.

In this context, it should be appreciated that one of the primary contributors to early mortality in cancer patients, regardless of tumor origin, is the collapse of the immune system.

Notably, patients with a low absolute lymphocyte count (ALC), defined herein as less than 1,000 cells/μL, are particularly susceptible to immune failure and diminished antitumor defense. In such cases, administration of N-803 in combination with at least one immunotherapy agent reactivates the immune system in a synergistic manner by promoting the expansion and functional activation of lymphocytes, including CD4+, CD8+, memory T cells NK cells, and monocytes. This immune system restoration leads to the establishment of a bioshield, which functions as a barrier to tumor progression. The bioshield enhances immune surveillance, improves recognition and elimination of cancer cells, and provides resistance to immune evasion strategies employed by tumors. Importantly, the inventors have observed that patients with previously suppressed immune systems show marked clinical improvement when this bioshield is established.

Furthermore, the therapeutic induction of a bioshield may significantly improve outcomes for patients who are non-responsive to standard immunotherapies, such as checkpoint inhibitors alone, or who have experienced immune exhaustion due to advanced disease or cytotoxic treatments. As such, the bioshield-based approach described herein offers a powerful and innovative strategy for improving survival and delaying disease progression across a broad range of cancer types.

In yet another aspect of the present disclosure, a method of treatment for a patient with lymphopenia is provided. The method comprises administering to the patient (a) an effective amount of an IL-15N72D:IL-15RαSu/Fc complex (N-803) formulated for administration to the patient, and (b) an effective amount of granulocyte colony stimulating factor (G-CSF) formulated for administration to the patient. In some embodiments, the patient has been or will be treated with chemotherapy. The chemotherapy may comprise at least one of a taxane, a platinum-based agent, cyclophosphamide, fludarabine, an m-TOR inhibitor, a tyrosine kinase inhibitor, doxorubicin, etoposide, ifosfamide, vinblastine, or methotrexate. The G-CSF may be administered before chemotherapy, after chemotherapy, or both before and after chemotherapy. In some embodiments, the patient has been or will be treated with radiotherapy (RT). The N-803 may be administered before RT, after RT, or both before and after RT. The patient may have cancer. The N-803 may be administered at a dose ranging from 10 to 50 μg/kg, including 15 μg/kg. The G-CSF may be administered at a dose ranging from 1 to 100 μg/kg.

Furthermore, a method of providing a protective bioshield against cancer in a patient in need thereof is also provided, wherein the bioshield comprises an induced cytotoxic (CD8⁺) and helper (CD4⁺) T cell-mediated immune response to the cancer, the method comprising administering to the patient a combination of effective amounts of N-803 and G-CSF. In some embodiments, the method further comprises administration of at least one immunotherapy agent selected from the group consisting of: checkpoint inhibitors (CPI), NK cell therapies, m-ceNK cell therapies, t-haNK cell therapies, recombinant adenoviral vectors such as Ad5 expressing tumor-associated antigens, yeast-based vaccines or vectors, HDAC inhibitors, and cytokines such as interleukin-7 (IL-7) and interleukin-21 (IL-21). The N-803 may be administered to the patient via injection, such as subcutaneous or intravenous (IV) injection.

The G-CSF may also be administered via subcutaneous or IV injection.

A composition for use in the treatment of a patient with lymphopenia is further provided, the composition comprising (a) an effective amount of an IL-15N72D:IL-15RαSu/Fc complex (N-803) formulated for administration to the patient, and (b) an effective amount of granulocyte colony stimulating factor (G-CSF) formulated for administration to the patient. In some embodiments, the N-803 and G-CSF are formulated together for simultaneous administration to the patient. Alternatively, the N-803 and G-CSF may be formulated separately for either simultaneous or sequential administration to the patient.

Consequently, a kit for use in the treatment of a patient with lymphopenia is also contemplated, the kit comprising: (a) a first vial comprising an effective amount of an IL-15N72D:IL-15RαSu/Fc complex (N-803) formulated for administration to the patient, (b) a second vial comprising an effective amount of granulocyte colony stimulating factor (G-CSF) formulated for administration to the patient, (c) at least one bag containing a sterile saline solution, and (d) directions for use thereof. The N-803 and G-CSF may be formulated together for simultaneous administration to the patient, or formulated separately for either simultaneous or sequential administration.

Additionally, a method of treatment for a patient with lymphopenia is disclosed, the method comprising administering to the patient an effective amount of (1) IL-7 or a stabilized derivative thereof, (2) an IL-15N72D:IL-15RαSu/Fc complex (N-803) comprising a dimeric IL-15RαSu/Fc and two IL-15N72D molecules, and (3) G-CSF. In some embodiments, the patient has previously been treated with chemotherapy and/or radiotherapy. The chemotherapy may comprise at least one of a taxane, a platinum-based agent, cyclophosphamide, fludarabine, an m-TOR inhibitor, a tyrosine kinase inhibitor, doxorubicin, etoposide, ifosfamide, vinblastine, or methotrexate. The patient may have cancer or may be concurrently undergoing treatment with chemotherapy and/or radiotherapy.

Consequently, the inventors also contemplate augmentation of N-803 therapy for subjects with cancer treatment failure and lymphopenia with co-administration of G-CSF, alone or in combination with cell-based therapy, checkpoint inhibitor therapy, cancer vaccine therapy, and/or chemotherapy. Suitable forms of G-CSF include filgrastim optionally pegylated), recombinant human granulocyte colony stimulating factor (rhG-CSF), r-metHuG-CSF, and various filgrastim biosimilars. In a phase 3 clinical trial involving patients with small cell lung cancer, subcutaneous (SC) administration of NEUPOGEN® at doses ranging from 4 to 8 mcg/kg/day on days 4 through 17 of the chemotherapy cycle was evaluated in comparison to placebo. The results of this study showed that G-CSF administration significantly reduced the incidence of febrile neutropenia, decreased the need for hospitalization, and reduced the use of intravenous antibiotics. These findings support the use of G-CSF as an adjunct therapy to the treatments discussed herein to help maintain chemotherapy dose intensity and reduce the risk of infectious complications. While the G-CSF study did not demonstrate a significant difference in overall survival or disease progression between the treatment and placebo groups, the clinical benefit of G-CSF in preventing febrile neutropenia and maintaining the continuity of chemotherapy regimens is well established and deemed beneficial in conjunction with the teachings presented herein.

For example, in the context of the above, G-CSF is administered in combination with IL-15-based immunotherapeutics, such as the IL-15N72D:IL-15RαSu/Fc complex (N-803), to synergistically enhance immune recovery and immune function following chemotherapy and/or radiotherapy. The combined use of G-CSF and N-803 promotes the recovery of neutrophils while concurrently stimulating cytotoxic immune cells, thereby offering a dual benefit of hematologic support and immunotherapeutic potentiation. The administration of G-CSF in such methods may be before, after, or concurrently with chemotherapy and/or radiotherapy, depending on the specific treatment protocol and clinical condition of the patient.

Further aspects, details, and considerations for use in conjunction with the teachings presented herein are described in US 2021/0188933, which is incorporated by reference herein.

Embodiments of the present disclosure are further described in the following examples.

However, it should be noted that the examples are merely illustrative and do not in any way limit the scope of the invention as claimed.

### Examples

N-803 has been shown to proliferate and activate NK cells, and to a lesser degree also CD4+, CD8+, and memory T cells, without upregulating Treg cells. Based on this mechanism of action, the inventors contemplate use of N-803 as a therapeutic agent with the ability to reverse or prevent lymphopenia in subjects with cancer, and especially in subjects with cancer that is treated with one or more immune therapeutic agents but where such treatment induces or exacerbates (or has induced or has exacerbated) lymphopenia. Conventionally, such treatment with immune therapeutic agents is discontinued to avoid worsening of lymphopenia.

Unfortunately, however, discontinuation of immune treatment is typically associated with increased mortality.

The inventors have now discovered that treatment with one or more immune therapeutic agents can be initiated and/or continued upon diagnosis of lymphopenia, even where such treatments are known to cause or worsen lymphopenia. Beneficially, and as is shown in the examples below, use of N-803 in such patient population will not only address lymphopenia, but also significantly increase mean overall survival in a variety of cancers.

NSCLC (N-803 in First Line Treatment with CPI)

N-803 acts as a lymphocyte stimulating agent in cancer patients: To confirm the contribution of effect of N-803 as a lymphocyte stimulating agent, as previously shown in healthy volunteers, analysis was performed in patients with NSCLC (non-small cell lung cancer) as first line treatment with CPI (checkpoint inhibitor) treatment.

To that end, the inventors queried clinical data from QUILT-2.023 (Clinical Trial ID: NCT03520686, incorporated by reference herein), an open-label randomized controlled study in first-line, stage III/IV NSCLC of checkpoint inhibitor (CPI) therapy alone compared to CPI+N-803 for participants with PD-L1 ≥1%. Absolute lymphocyte count (ALC) from a complete blood count (CBC) was assessed over 3-week cycles of therapy. Statistical analysis to compare ALC values over time was performed using a mixed model for repeated measures of actual ALC values (effects for baseline ALC value, treatment group, time, and group by time interaction); the P-value is from the type III test for the fixed effect of treatment group.

Before treatment, the baseline mean ALC for the groups was similar, being 1.64 cells/mL (Range 0.50-3.69) for CPI and 1.786 cells/mL (Range 0.60-3.69) for CPI+NAI. ALC over time for CPI+NAI therapy was elevated and significantly higher compared to CPI alone over the first 10 cycles of therapy (P=0.0065). Analysis of ALC over the first 10 cycles of treatment demonstrated the ability of N-803 to markedly increase ALC soon after starting therapy and to sustain those increases over time, even in the presence of CPI treatment that is known to reduce ALC and to substantially contribute to lymphopenia. While prior data confirmed that N-803 is the first cytokine fusion molecule in its class to treat lymphopenia in normal healthy volunteers not undergoing CPI treatment, N-803 unexpectedly was capable to prevent/reverse lymphopenia over a large number of treatment cycles with CPIs. This potential of reversing lymphopenia induced by treatment, and prolonging survival, and improving prognosis across various tumor types as further shown below, presents a possible paradigm change in cancer care.

N-803 reverses lymphopenia and prolongs overall survival in relapsed cancer patients with prior CPI treatment: To further investigate whether or not the increase and maintenance of ALC would have a clinically meaningful outcome, the inventors further queried clinical data from a clinical trial in NSCLC with N-803+CPI in patients who relapsed prior CPI treatment.

Multiple Phase 1B and two Phase 2 trials were completed with a total of 98 NSCLC patients enrolled to study the combination of NAI with ICI in patients who relapsed on CPI. The QUILT-3.055 study (Clinical Trial ID: NCT03228667) demonstrated that median overall survival (mOS) was significantly prolonged in 86 patients who were progressing on CPIs and received the same CPI with no intervening therapy other than the addition of N-803 as compared to the multiple historical studies of docetaxel in this setting.

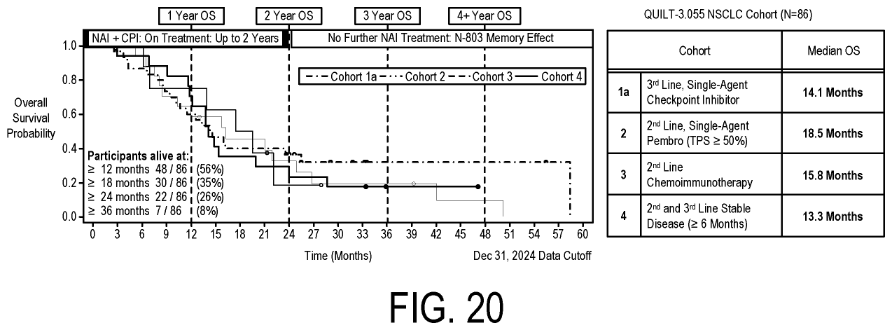

FIG. 20 depicts a Kaplan Meier plot for this outcome. As can be seen from FIG. 20, in 2nd and 3rd line NSCLC patients who relapsed on checkpoint inhibitors, the administration of N-803 and the same checkpoint inhibitor without any intervening therapy during the period of progression, resulted in recovery of response to the checkpoint inhibitor with the regeneration of lymphocytes and the upregulation of MHC-I, resulting in overall survival prolongation (longer than current standards of care of chemotherapy (docetaxel ~7.0 months). The impact of the level of ALC and the differences in ALC levels at less than median of <1,200 lymphocytes per microliter compared to the baseline median greater than ≥1,200 was examined to determine the impact of ALC on overall survival. The demonstration that N-803 regenerates lymphocytes and sustains this therapeutic effect through the treatment with resultant prolongation of overall survival is provided below.

Table 1 depicts further exemplary results for overall survival (overall survival by baseline median absolute lymphocyte count (ALC)) and the impact of absolute lymphocyte count levels on prolonging overall survival in subjects relapsed on CPI.

TABLE 1

| | Baseline ALC < | Baseline ALC ≥ | | |
|---|---|---|---|---|
| | Median (1,200) | Median (1,200) | | Hazard Ratio |
| Variable | N = 42 | N = 44 | P-Value | (95% CI) |
| Number of Deaths | 35 (83%) | 28 (64%) | | |
| Median Survival (months) | 11.7 | 21.1 | 0.0005 | 0.40 (0.24, 0.68) |
| 95% CI for the Median Survival | 8.5, 14.6 | 13.9, 42.1 | | |

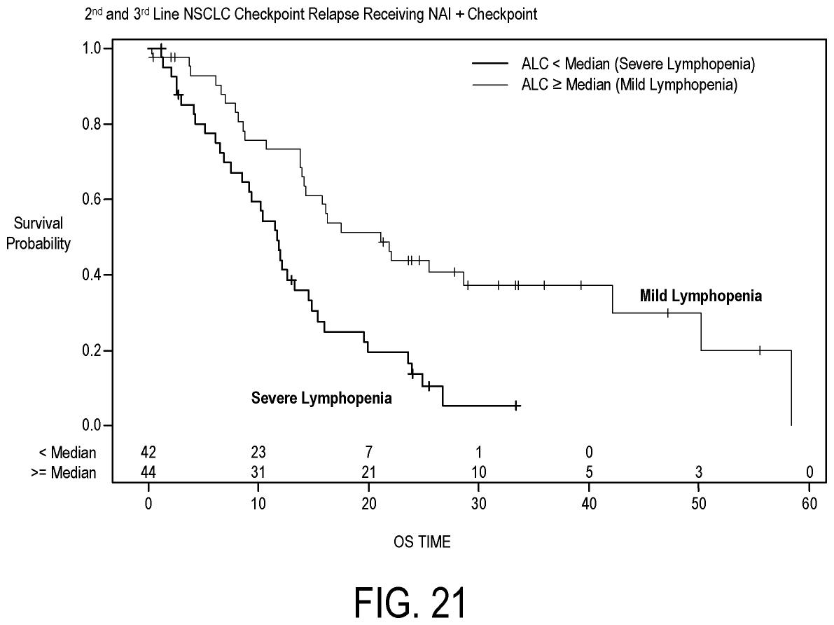

As can be seen from the data in Table 1, these findings confirm that improving lymphopenia indeed significantly prolonged overall survival. The mOS (mean overall survival) in subjects with baseline ≥1,200 lymphocytes per microliter was 21.1 months (95% CI: 13.9, 42.1), far exceeding the historical OS of subjects who had relapsed on chemo-immunotherapy including CPI when given standard of care docetaxel, in which the mOS is approximately 7.0 months. The Kaplan-Meier analysis demonstrates maturity of the data and the significant prolongation of OS in subjects with ALC greater than the median of ≥1,200 lymphocytes per microliter at baseline as is shown in FIG. 21, showing overall survival by baseline ALC median for NSCLC subjects.

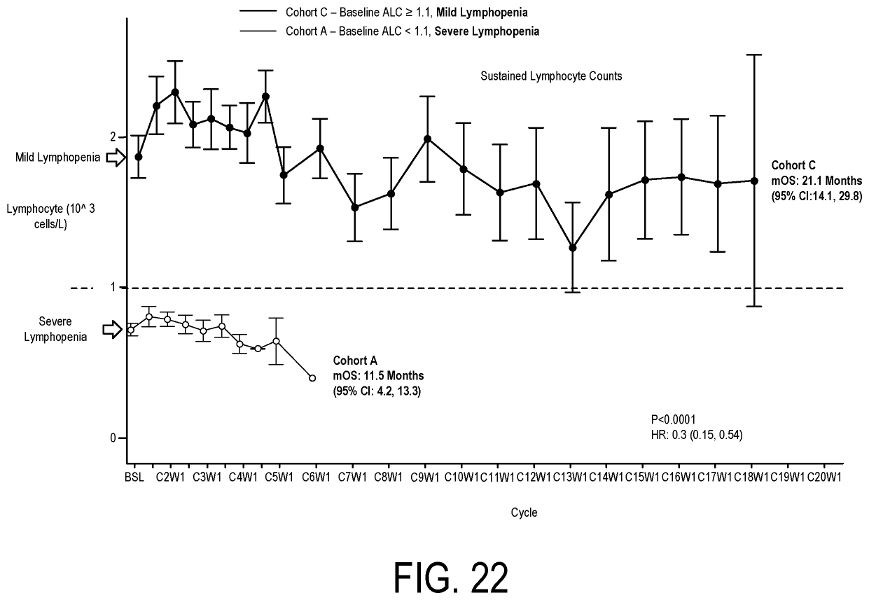

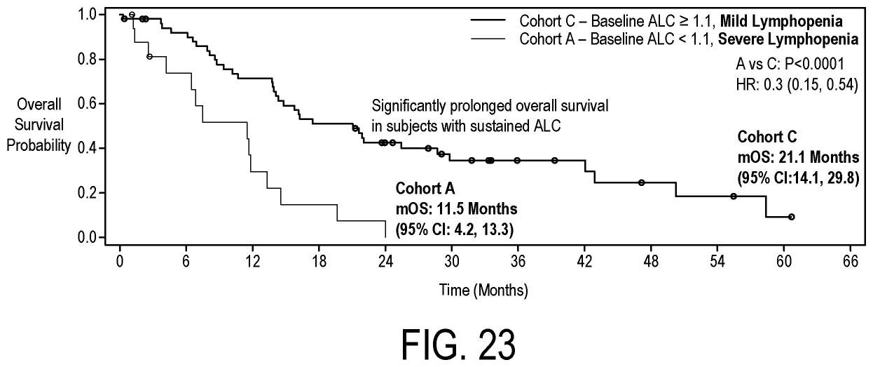

To examine the impact of ALC on prolonging overall survival when sustained by N-803 through the course of therapy, the therapeutic effect of N-803 plus checkpoint inhibitor was studied in the combined Phase 2 cohorts of NSCLC subjects who relapsed on checkpoint inhibitor (N=98). The results showed consistency with that of QUILT-88 (Clinical Trial ID: NCT04390399) in metastatic pancreatic cancer where N-803 was combined with cell therapy (PD-L1 t-haNK). The therapeutic effect of N-803 in sustaining lymphocyte counts in subjects with 2nd and 3rd line NSCLC and the contribution of effect of ALC in prolonging overall survival is shown in FIG. 22 (showing therapeutic effect of N-803 in NSCLC Patients with sustained lymphocyte count in subjects with severe and mild lymphopenia following CPI relapse). Here, patients with baseline severe lymphopenia (<1,100 lymphocytes per microliter, N=17) were compared to those with a baseline mild lymphopenia (≥1,100 lymphocytes per microliter, N=52). The difference in overall survival was highly significant with a median overall survival of 11.5 months (95% CI: 4.2, 13.3) in the severe lymphopenia group (<1,100) and median overall survival of 21.1 months (95% CI: 14.1, 29.8) in the mild lymphopenia group (≥1,100) with a P-value: <0.0001, HR: 0.3 (0.18, 0.54). FIG. 23 shows a Kaplan Meyer plot illustrating the contribution of effect of ALC (Sustained by N-803) with prolonged overall survival in NSCLC subjects with lymphopenia. These findings confirm that chemo-immunotherapy adversely affects the immune system resulting in severe lymphopenia as a consequence of their cancer and the prior therapy, with consequences of a poorer prognosis in terms of overall survival. This study demonstrates that N-803 has the capability to sustain lymphocyte levels as shown in the subjects with mild lymphopenia and achieve significantly higher median overall survival of 21 months, significantly higher than standard of care docetaxel (~7 months OS). FIG. 23 summarizes the finding that N-803 can sustain lymphocyte proliferation and that by maintaining lymphopenia as close to normal, overall survival can be significantly improved.

mPCa (N-803 in >3^{rd} Line Treatment with t-haNK)

Lymphopenia rescue (‘Cancer BioShield’) results in significantly prolonged overall survival following N-803 and PD-L1 t-haNK Chemo-Immunotherapy in 3^{rd} to 6^{th} Line Metastatic Pancreatic Cancer Patients: As is shown in more detail below, the inventors have demonstrated that the in vivo protection of lymphocytes (via subcutaneous injection of N-803) and the ex-vivo protection of the collapse of the immune system (via infusion of PD-L1 t-haNK) delivers significant benefits to the treatment of cancer. In this context, it should be appreciated that the fundamental root cause of early death in patients with cancer across all tumor types is the collapse of the immune (lymphocyte) system and that cancer is a symptom of this collapse. Viewed from a different perspective, the inventors postulate that survival is closely associated with lymphopenia and ALC, with patients who develop lymphopenia due to the cancer itself, or as a consequence of standard-of-care radiation (e.g., SBRT) or chemotherapy, having shorter survival than those with ALC in the normal range.

Lymphopenia (ALC<1,000), a high neutrophile-to-lymphocyte (NLR) ratio, and high CA19-9 levels are generally associated with poor prognosis in pancreatic cancer. As is shown in more detail below, N-803 reverses and at least partially prevents lymphopenia and prolongs overall survival in patients with advanced pancreatic cancer, even those who have failed all standards of care.

Programmed death-ligand 1 (PD-L1) targeting high-affinity NK (PD-L1 t-haNK) are engineered to express high-affinity CD16 and PD-L1 specific chimeric antigen receptor (CAR-NK). These ex vivo generated lymphocytes, when given in combination with N-803 (in vivo stimulation of lymphocytes), target immunosuppressive myeloid derived suppressor cells (MDSCs) to augment tumor response and patient outcomes. By overcoming immune evasion and rescuing the treatment-induced collapsed immune system, prolonged overall survival can be achieved.

In particular, QUILT-88 (NCT04390399, incorporated by reference herein in its entirety) was a phase 2 open-label multicenter study wherein cohort C patients with locally advanced or mPC who had progressed after 2^{nd} to 6^{th} line therapy received SBRT, low-dose chemotherapy, N-803, and PD-L1 t-haNK cells. Median overall survival (mOS), CA19-9 (an indicator of tumor burden) ALC and NLR levels were assessed. Associations between these parameters were determined, specifically between mOS for those with ALC< vs. ≥the on-treatment median (580 cells/μL); for those with CA19-9< or ≥the baseline median (3,852 U/mL); and for those who overcame severe lymphopenia. In addition, mOS by the neutrophil-to-lymphocyte (NLR) ratio was analyzed to examine the effect of overcoming high immunosuppressive neutrophils and overcoming lymphopenia.

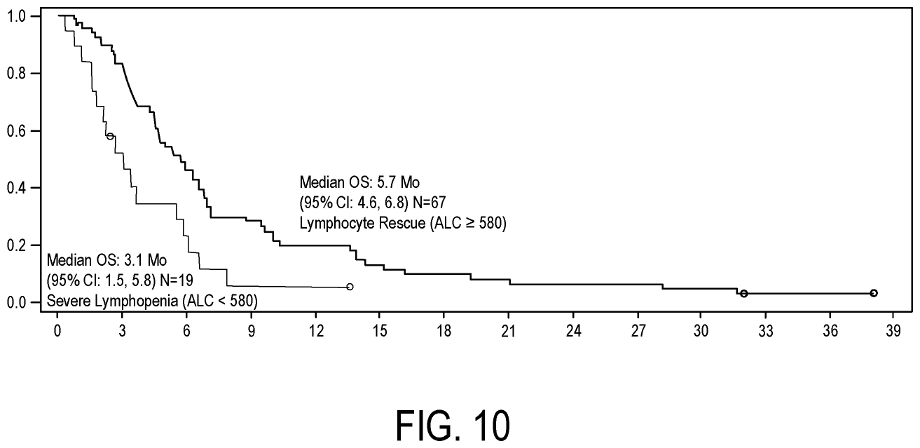

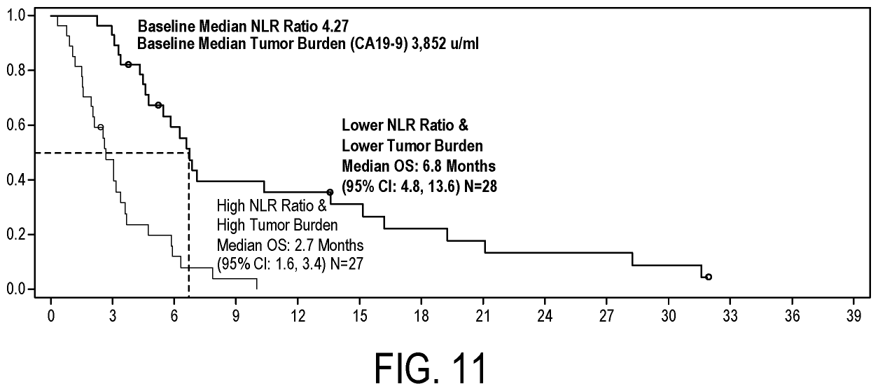

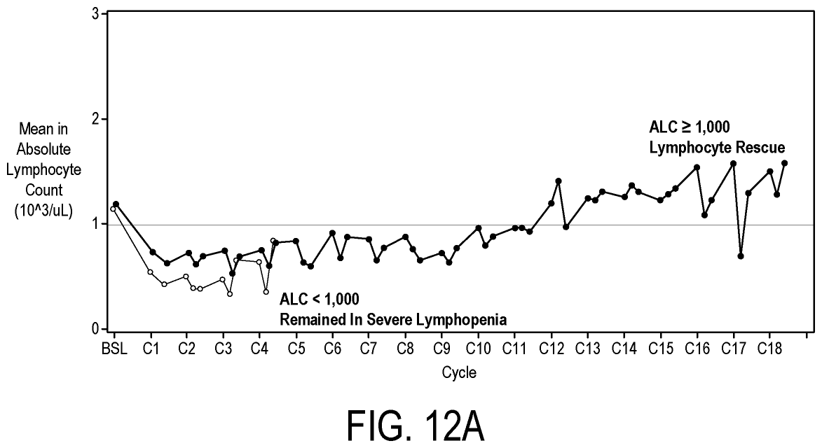

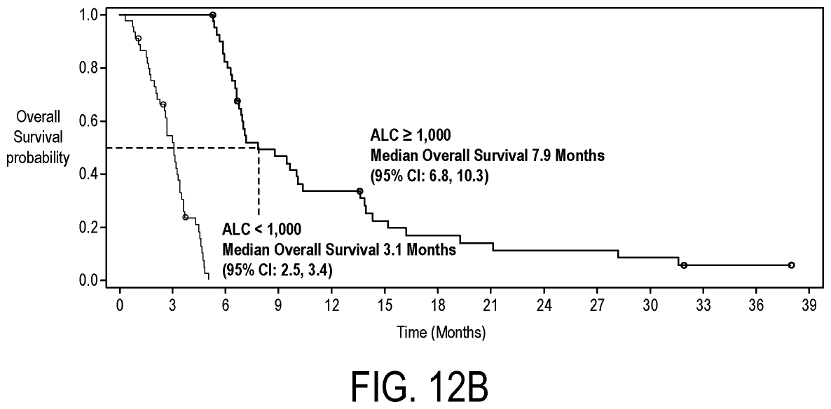

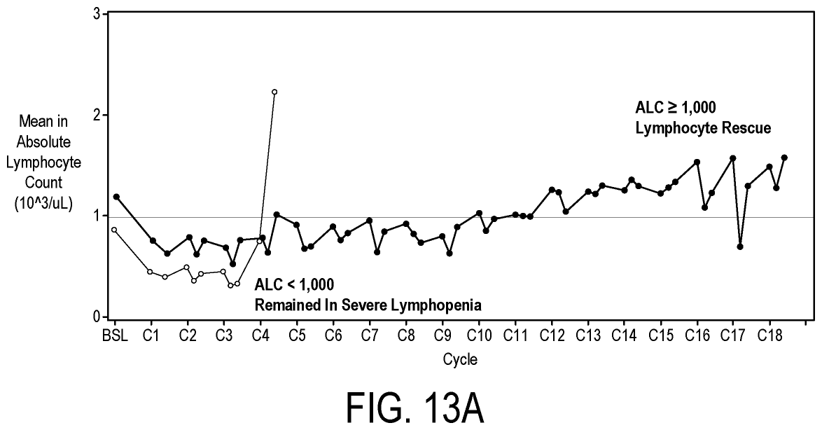

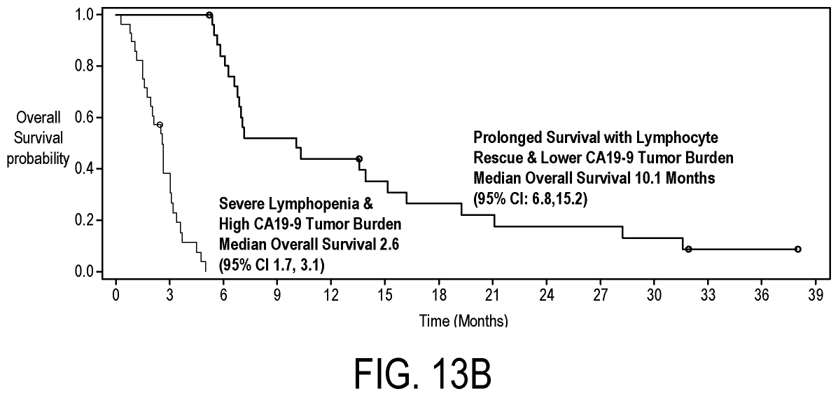

In this study, the inventors found that significant prolonged survival was associated with lymphopenic rescue (as measured by ALC and shown in FIG. 10. Significant prolonged survival was also associated with the reduction of Neutrophil-to-Lymphocyte Ratio (NLR) and tumor burden (CA19-9) as is shown in FIG. 11. Further prolonged survival was associated with level of lymphocyte (ALC) rescue achieving ALC≥1,000, independent of CA19-9 tumor burden as can be taken from FIG. 12. Further prolonged survival was associated with level of lymphocytes (ALC) and CA19-9 tumor burden as is shown in FIG. 13. Once more, as can be seen from the data, N-803 improves ALC and reduces NLR: Reversal of lymphopenia with higher ALC and lower NLR is associated with significant prolonging of overall survival in patients with pancreatic cancer. Achieving ALC≥1,000 further prolongs survival suggesting the benefit of increasing ALC. In addition, it should be appreciated that administration of PD-L1 t-haNK (CAR-NK) overcomes immunosuppressive MDSCs and plays a role by providing an exogenous source of active NK cells.

mPC (N-803 in >3^{rd} Line Treatment with t-haNK, Stratified by CA19-9)

Lymphopenia rescue and lower CA19-9 levels are associated with prolonged overall survival following N-803 and PD-L1 t-haNK chemo-immunotherapy for ≥3rd line greater metastatic pancreatic cancer (mPC): Lymphopenia and high CA19-9 levels are generally associated with poor prognosis in pancreatic cancer. The inventors postulated that N-803 has the potential to rescue lymphopenia, especially in a patient population with lower tumor burden. Programmed death-ligand 1 (PD-L1) targeting high-affinity NK (PD-L1 t-haNK) are engineered to express high-affinity CD16, endoplasmic reticulum-retained IL-2, and a PD-L1 specific chimeric antigen receptor (CAR). These cells, when given in combination with N-803, may be able to target immunosuppressive MDSCs to augment tumor response and patient outcomes, while at the same time support NK-cell based immune therapy and restore at least partially the innate immune system.

Here, as is described in QUILT-88 (NCT04390399, incorporated by reference herein in its entirety) a phase 2 open-label multicenter study was conducted in which cohort C patients with locally advanced or mPC who had progressed after 2^{nd} to 6^{th} line therapy received SBRT, low-dose chemotherapy, N-803, and PD-L1 t-haNK cells. Median Overall Survival (mOS), CA19-9 (an indicator of tumor burden) and ALC levels were assessed. Associations between these parameters were determined, specifically between mOS for those with ALC< vs. ≥the on-treatment median (580 cells/μL); for those with CA19-9< or >the baseline median (3,852 U/mL); and for those survived >5 months and CA19-9 of either < or ≥the baseline median (1,516 U/mL). In addition, mOS by the neutrophil-to-lymphocyte (NLR) ratio was analyzed to examine the effect of relatively high immunosuppressive neutrophils with relatively low lymphocytes.

The inventors found that survival was associated with ALC. Indeed, the mOS for all ALC<580 cells/μL (on-treatment median) of 3.1 months (95% CI 1.5, 5.8) was significantly lower than the 5.7 months (95% CI 4.6, 6.8) for at least 1 ALC≥580 cells/μL (P-value 0.005; HR 0.46 (0.26, 0.80)) as is also shown in Table 2.

TABLE 2
On-Treatment ALC & Survival in QUILT-88 Pancreatic Cancer Participants

| | | All ALC < mALC | At Least 1 ALC ≥ | Log- | |
|---|---|---|---|---|---|
| | All | (580 cells/μL) | mALC (580 cells/ | Rank | |
| Variable | (N = 86) | (N = 19) | μL) (N = 67) | P-value | HR (95% CI) |
| Deaths, n (%) | 78 (91%) | 17 (89%) | 61 (91%) | | |
| mOS, mo. (95% CI) | 5.0 (3.7, 6.3) | 3.1 (1.5, 5.8) | 5.7 (4.6, 6.8) | 0.005 | 0.46 (0.26, 0.80) |

ALC—Absolute Lymphocyte Count;
CI—Confidence Interval;
mALC—median on-treatment ALC;
mOS—median overall survival;
mo.—months;
HR—hazard ration

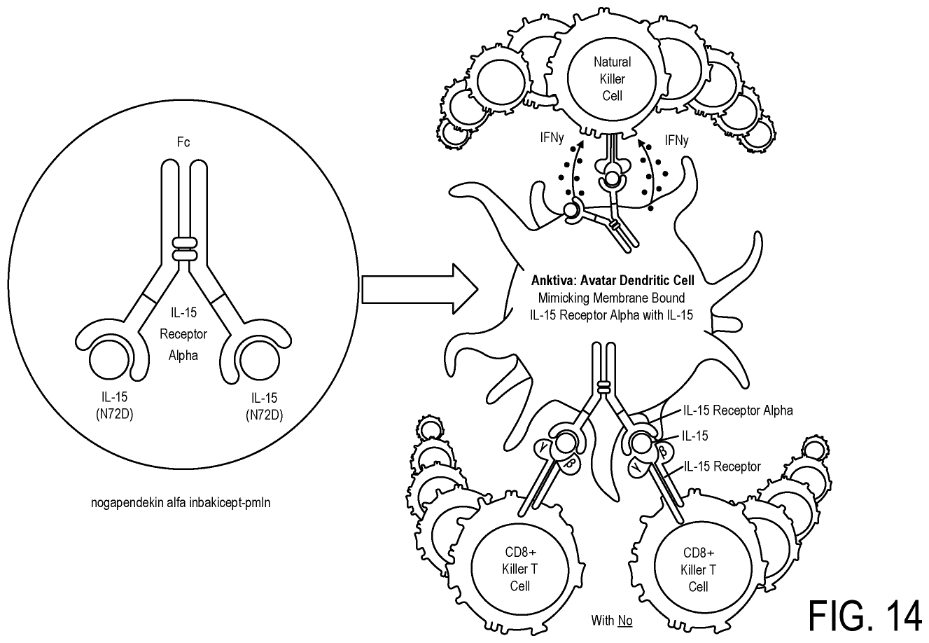

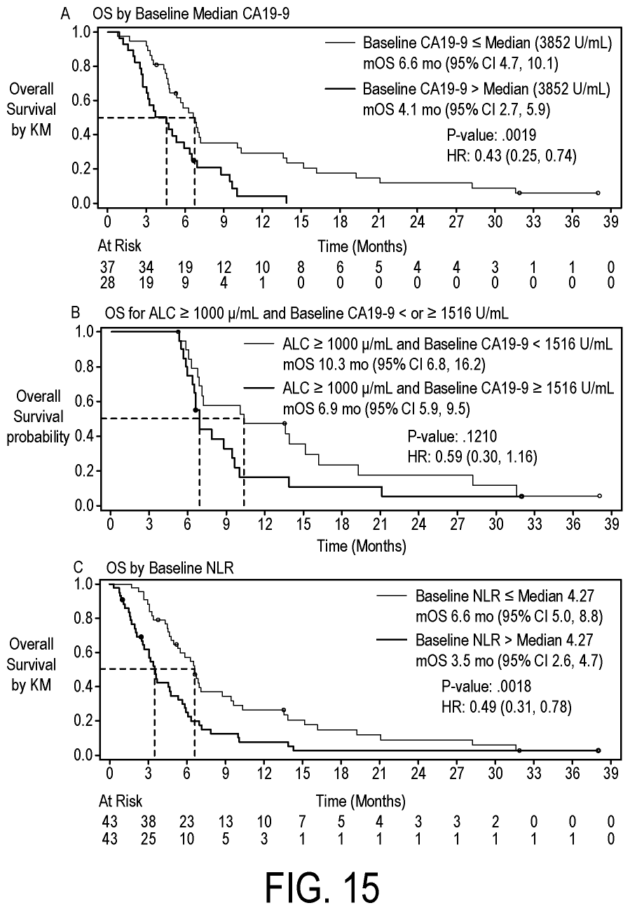

The inventors further found that survival was associated with CA19-9 as can be readily seen from FIG. 14. The mOS of 6.6 months (95% CI 4.7, 10.1) for CA19-9≤3,852 U/mL (baseline median) was significantly longer than the 4.1 months (95% CI 2.7, 5.9) for >3,852 U/mL (P-value 0.0019; HR 0.43 (0.25, 0.74)) as can be seen from FIG. 15 (top panel). The inventors also found that survival was associated with ALC and CA19-9. Participants who achieved ALC≥1,000 cells/μL survived >5 months (median survival time). mOS for participants survived ≥5 months was higher at 10.3 months (95% CI 6.8, 16.2) when the CA19-9 level was <1,516 U/mL (median baseline level for these participants) than the 6.9 months (95% CI 5.9, 9.5) for CA19-9≥1516 U/mL, but the difference was not significant (P-value 0.1210; HR 0.59 (0.30, 1.16)). See also FIG. 15, middle panel. Finally, the inventors found that survival was associated with the NLR. mOS was 6.6 months (95% CI 5.0, 8.8) for participants having a NLR≤4.27 (baseline median) and 3.5 months (95% CI 2.6, 4.7) for NLR>4.27 (P-value 0.0018; HR 0.49 (0.31, 0.78)) as is shown in FIG. 15, bottom panel.

Higher ALC, and lower CA19-9 and NLR, were associated with prolonged survival, suggesting the benefit of increasing ALC. N-803, the first therapy to act as a lymphocyte-stimulating agent (LSA) by proliferating NK and CD8+ T cells, increases ALC. Moreover, addition of PD-L1 t-haNK to the therapeutic regimen plays a role in overcoming the deleterious effects of lymphopenia by providing an exogenous source of active NK cells.

Therefore, and viewed from a different perspective, it should be appreciated that treatment of 3rd line or greater metastatic pancreatic cancer with N-803 plus off-the-shelf NK-92 cells targeted to PD-L1 (PD-L1 t-hank) demonstrated overall survival benefit. This is particularly relevant as pancreatic cancer has claimed an estimated 47,000 lives in the USA in 2020, with an expected median overall survival of 3 months in the 3rd line setting. The results of the QUILT-88 study showed that the overall survival rate for patients continued to be double compared to historical survival rates after two or more prior lines of therapy. The median OS in this highly advanced group of patients, up to seven lines (N=83) of treatment, was 5.8 months (95% CI: 4.9, 6.4 months), exceeding the approximately 2- to 3-month historical median OS. In the 3rd line setting (N=41), the median OS in this group was 6.3 months (95% CI: 5.0, 7.2 months), more than doubling the historical OS.

Moreover, the baseline median CA 19-9 level (a marker of metastatic pancreatic disease) of the enrolled subjects (N=83) was very high at 4120 IU/mL, a significant increase from normal levels of 40 IU/ml. In subjects with CA 19-9 levels less than 4120 IU/mL (N=40), the median OS was 6.9 months (95% CI: 5.7,10.9). In light of the heavy tumor burden associated with patients with CA19-9 levels as high as 4,120 IU/ml, the inventors performed an analysis to compare the overall survival rate between those subjects with levels lower than 4,120 IU/mL to those greater than ≥4,120 IU/mL. To examine the impact of severe versus mild lymphopenia at baseline pre-treatment with N-803 and PD-L1 t-haNK as well as the impact of tumor burden, the patients in ≥3^{rd} line metastatic pancreatic cancer were stratified as shown in Table 3 below. The data demonstrated that severe lymphopenia is a poor prognostic factor and that ALC at baseline, together with lower tumor burden (CA19-9) contributes to overall survival effect. Table 3 shows Overall Survival by Baseline Median Absolute Lymphocyte Count (ALC) and Median CA19-9.

TABLE 3

| | Baseline CA19-9 ≥ | Baseline CA19-9 < | | |
|---|---|---|---|---|
| | Median (4080) | Median (4080) | | |
| | and Baseline ALC < | and Baseline ALC ≥ | | |
| | Median (1045) | Median (1045) | | |
| | Severe Lymphopenia | Mild Lymphopenia | | Hazard Ratio |
| Variable | N = 25 | N = 24 | P-Value | (95% CI) |
| Number of Deaths | 24 (96%) | 21 (88%) | | |
| Median Overall Survival (months) | 3.1 | 7.1 | 0.0001 | HR: 0.29 (0.15, 0.56) |
| 95% CI, Median Survival | 1.9, 5.0 | 5.0. 10.9 | | |

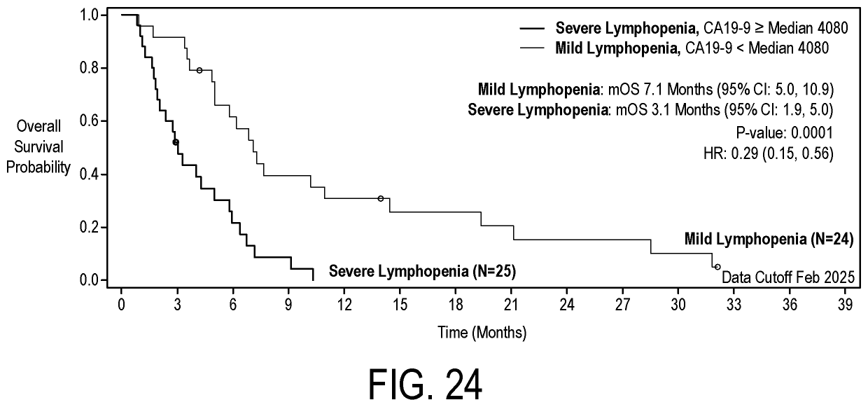

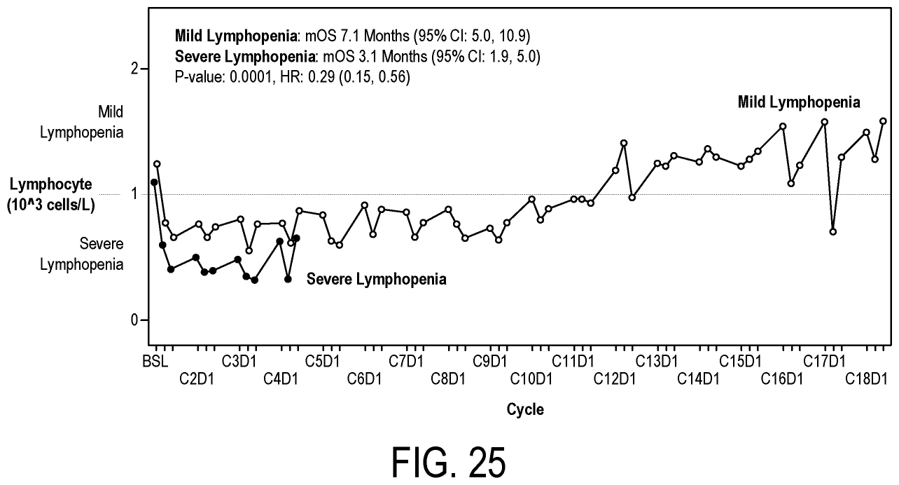

These findings confirmed that at baseline levels of severe lymphopenia combined with high tumor burden (CA19-9) the overall survival is 3.1 months. On the other hand, when baseline lymphocyte count is at a median ≥1045 lymphocytes per microliter, and a tumor burden of CA19-9<4080, the overall survival is significantly prolonged at 7.1 months (95% CI: 5.0, 10.9. P-value: 0; 0001, HR 0.29 [0.15, 0.56]). The Kaplan-Meier (KM) analysis demonstrates the significant prolongation of OS in subjects with ALC greater than the median of >1,045 lymphocytes per microliter at baseline. FIG. 24 is a Kaplan Meyer plot showing Overall Survival by Median Baseline ALC (Mild and Severe Lymphopenia) in ≥3rd Line Metastatic Pancreatic Subjects Stratified by CA19-9 Levels. FIG. 25 is a graph depicting therapeutic effect of N-803 in sustaining lymphocyte count in subjects with metastatic pancreatic cancer.

N-803 sustained ALC levels over time: As shown in the healthy volunteer studies of N-803, the proliferative effect of N-803 maintaining and sustaining the tissue effect of proliferating NK and T cells (lymphocytes) was confirmed by the temporal analysis of ALC levels across time. As shown in FIG. 25, the ALC levels ≥median (1,045 lymphocytes per microliter) is sustained throughout the course of therapy. Of note, this sustained effect of NAI combined with PD-L1 t-haNK recovers the subjects from severe lymphopenia into the range of mild lymphopenia and the resultant overall survival rate was significantly higher than those subjects with impaired immune system from the prior chemotherapy. This supports the potential for a CBC differential analysis to provide therapeutic biomarker of N-803 with baseline and sustained ALC treating lymphopenia as an indication of improved efficacy.

Solid Tumors (N-803 with Memory-Like Cytokine-Enriched Natural Killer (M-CENK) Cells in Locally Advanced or Metastatic Solid Tumors (Cancer Bioshield)

Lymphopenia, characterized as low Absolute Lymphocyte Count (ALC), defined as below 1,000 lymphocyte cells/μL in blood. Currently, no treatment for lymphopenia exists. Low levels of lymphocyte subsets of NK cells and T cells contribute to poor prognosis and early mortality in patients with cancer. Lymphopenia and treatment induced lymphopenia (from chemotherapy, radiation, and immunotherapy) has been poorly recognized since no treatment to reverse lymphopenia existed to date until the present disclosure of N-803, an IL-15 superagonist (in-vivo component) and infusion of M-ceNK (ex-vivo component).

For the first time, the in-vivo protection of lymphocytes (via subcutaneous injection of NAI) and the ex-vivo protection of the collapse of the immune system (via production and administration of M-CENK), presents a paradigm change in the treatment of cancer. Based on the data above and additional studies, the inventors have observed that a fundamental root cause of early death in patients with cancer across all tumor types is the collapse of the immune system and that cancer is a symptom. In this view, lymphopenia is more proximal to the disease and cancer is a more distal symptom. Survival is closely associated with lymphopenia and ALC, with patients who develop lymphopenia due to the cancer itself, or as a consequence of standard-of-care radiation (SBRT) or chemotherapy, having shorter survival than those with ALC in the normal range.

Natural Killer (NK) cells, key elements of lymphocytes subsets, are the immune system's first line of defense against infection and transformed cells that may progress to cancer if they evade immune surveillance. Memory-Like Cytokine-Enriched NK (M-CENK) cells generated from the patient's own PBMC-derived NK cells stimulated ex-vivo with IL-12, IL-18, and IL-15 superagonist N-803, overcome the collapse of the immune system across all tumor types. M-CENK cells express elevated IFN-γ and granzyme B compared to healthy donor NK cells, and display toxicity against multiple tumor cell lines.

In a Phase 1 First-in-Human Study QUILT-3.076 (NCT04898543, incorporated by reference herein in its entirety) safety and preliminary efficacy of M-CENK cells plus N-803 in participants with locally advanced or metastatic solid tumors was assessed as follows: Cohort 1 (up to N=40) enrolled participants with newly diagnosed, 1st line solid tumors. Cohort 2 (up to N=21) enrolled participants with relapsed or refractory solid tumors who progressed after >2 lines of therapy. M-CENK cells were administered by infusion (N=10 Enrolled to Date) weekly up to 10 times and N-803 SC for up to 5 doses every 2 weeks prior to every other dose of M-CENK cells. Patient infusions to date: N=10 (as of May 2025). All infusion were performed in an outpatient setting. Cancer types (2nd line & greater): Breast (N=4), Colon (N=1), Duodenum (N=1), Renal (N=1), Pancreatic (N=1), Rectal (N=1), Osteosarcoma (N=1). Range of M-ceNK infusions (2 to 5 bags infused) with N-803 subcutaneously. Safety: Zero (0%) TRAE Grade 4 or 5. Zero (0%) cytokine storm. In this investigation, it is expected that treatment response and overall survival will follow the restoration or increase of ALC to normal levels.

### Solid Tumors (N-803 Alone or in Combination Immune Therapy)

In further studies, the inventors have shown the reversal of lymphopenia as assessed by lymphocyte count and by percentage change of ALC (absolute lymphocyte count) from baseline across multiple tumor types (N=265) when ALC≥1,000 is reached. The baseline median ALC and change over time (by ALC and percentage change) was measured across all subjects for all cycles of therapy to assess the reversal of lymphopenia with the administration of N-803 (Nogapendekin alfa inbakicept) in the following trials and across all histological types: QUILT-2.023 (NCT03520686, incorporated by reference herein): NSCLC, QUILT-3.076 (NCT04898543, incorporated by reference herein in its entirety): NSCLC, small-cell lung cancer (SCLC), urothelial, head and neck squamous, melanoma, renal, gastric, cervical, hepatocellular carcinoma (HCC), colorectal cancer, and QUILT-88 (NCT04390399, incorporated by reference herein in its entirety): Metastatic pancreatic cancer.

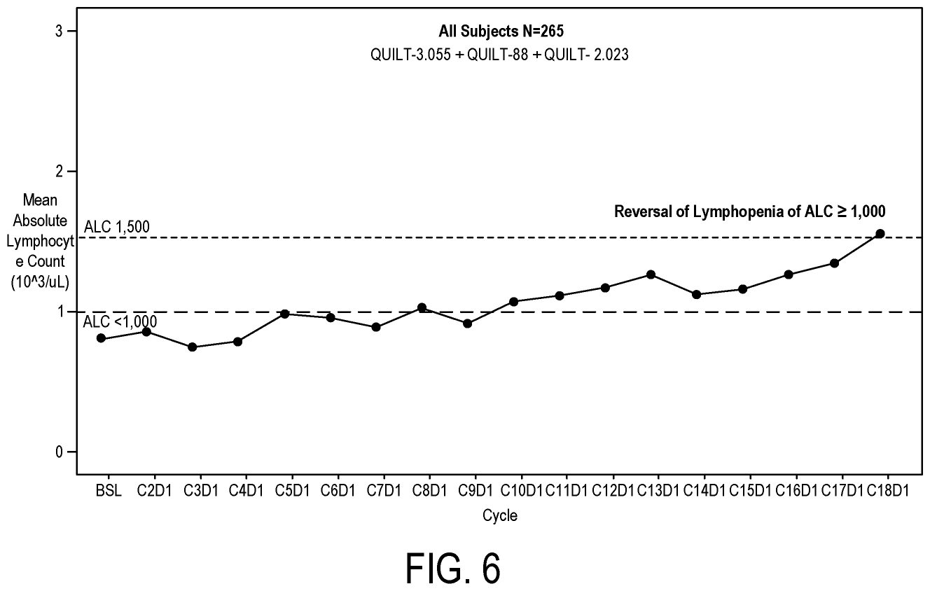

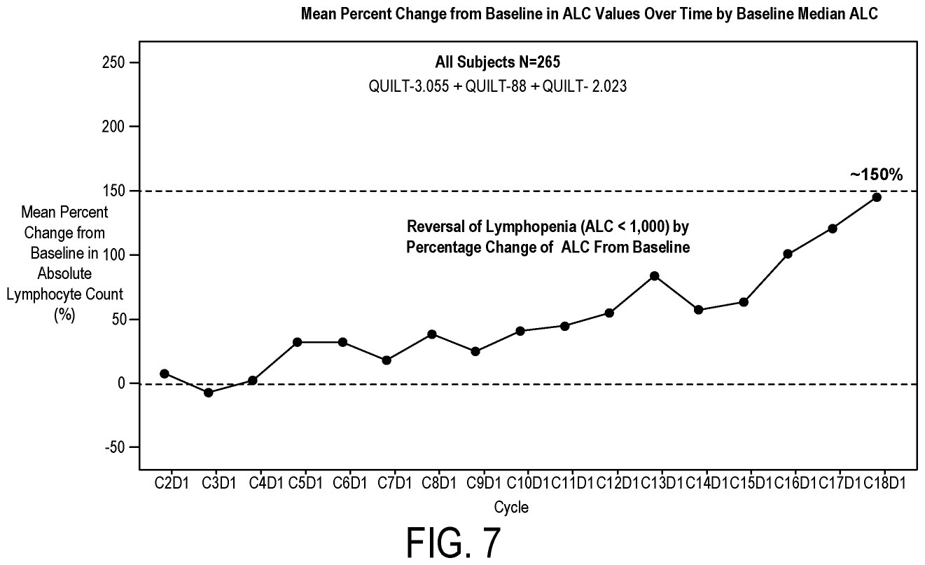

The combined findings from these studies demonstrate that N-803 acts as a Lymphocyte Stimulating Agent (LSA) and is tumor agnostic. In all subjects (N=265), throughout the duration of the therapy and regardless of the tumor histology, N-803 reversesed the ALC count above severe lymphopenia. FIG. 6 shows the increase by ALC, and FIG. 7 shows the percentage change from baseline of ALC over time and over all cycles. FIG. 6 illustrate that ALC increase over time from baseline lymphocyte count. In a tumor agnostic fashion, NAI reverses lymphopenia from an ALC<1,000 to an ALC approaching 1,500 lymphocytes per microliter. FIG. 7 illustrate that ALC increase over time from baseline by percentage change. In particular, N-803 increased the ALC over baseline and reached approximately 150% increase even in the presence of concomitant standard of care chemotherapy, radiation, and checkpoint inhibitors by cycle 18 and across all tumor types.

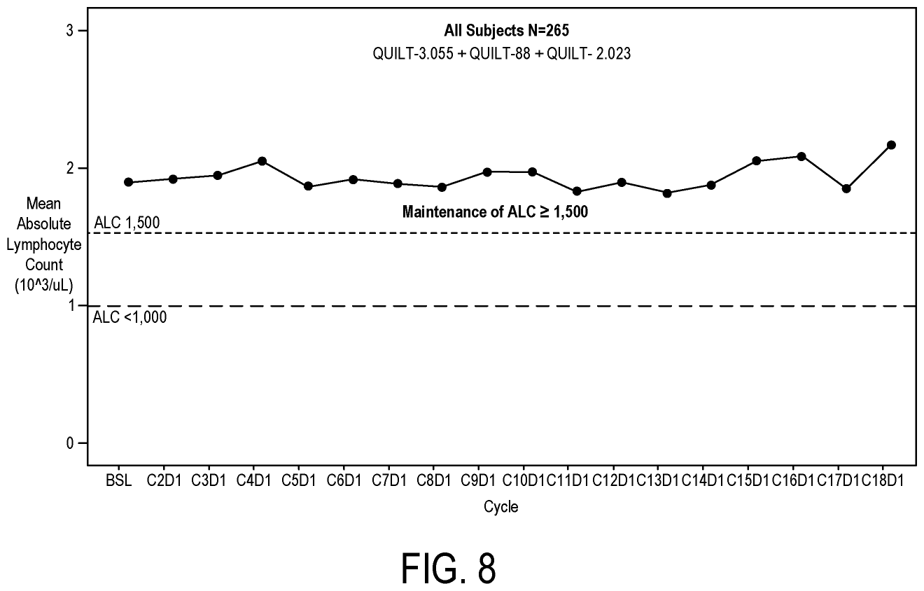

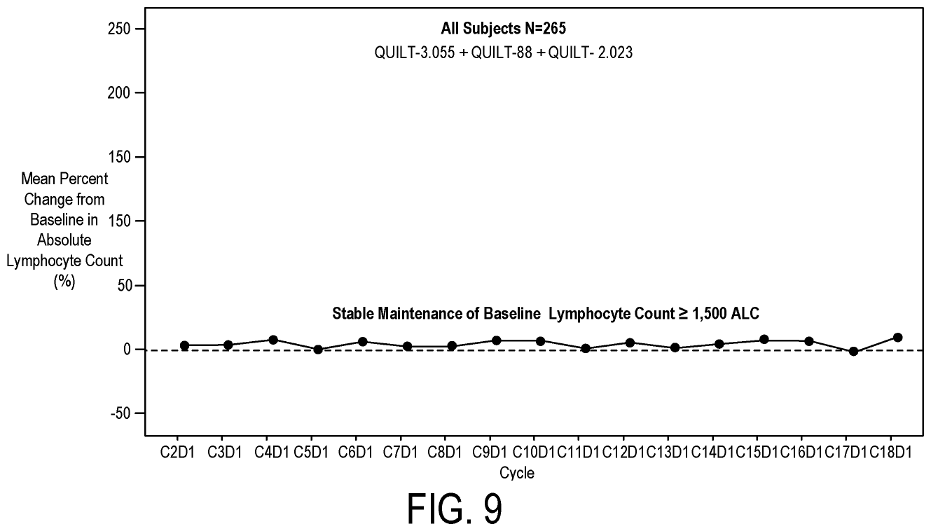

The inventors also unexpectedly found that N-803 maintained ALC levels at baseline beyond severe lymphopenia and sustained ≥1,500 across multiple tumor types even in the presence of concomitant chemotherapy, radiation and CPI standard of care. To demonstrate N-803's therapeutic efficacy as a lymphocyte stimulating agent to reverse and maintain ALC≥1,500 even in the face of adverse lymphopenic effects of chemotherapy, radiation, and/or immunotherapy, the ALC levels were measured in a pooled analysis (N=265) from QUILT-2.023, QUILT-3.055, and QUILT-88 in subjects with baseline ALC≥1,500. The results unexpectedly demonstrated that the ALC was maintained and sustained at that level with no decrease in lymphocytes despite the negative effects of chemotherapy and radiation on lymphocyte count. The ALC levels remained constant at the level greater than the average ALC≥1,500 across all cycles regardless of tumor type and regardless of standard of care therapy. These findings support the importance of N-803 in preserving lymphocytes and maintaining ALC to prevent lymphopenia. FIG. 8 (ALC by lymphocyte count over time) and FIG. 9 (percentage change of ALC over time) demonstrate stable and sustained levels of ALC across all cycles. Of note, N-803 stimulation of lymphocytes did not result in lymphocytosis above normal levels, further supporting the safety of N-803 as a lymphocyte stimulating agent.

Notably, prolonged overall survival in CPI failure was observed across multiple tumor types. Supportive data of the therapeutic effect of N-803 in prolonging overall survival by treating lymphopenia was provided by the study of N-803 combined with CPI in patients who relapsed on CPI across multiple tumor types. The following cohorts with advanced metastatic disease (98%) who were progressing on checkpoint inhibitor were studied (N=147): Non-Small Cell Lung Cancer (N=86), Small Cell Lung Cancer (N=10), Melanoma (N=15), Head and Neck Squamous Cell Carcinoma (N=14), Renal Cell Carcinoma (N=12), Other Solid Tumors (N=10) (Cervical, Gastric, Hepatocellular Carcinoma, Urothelial, Other) from QUILT-3.055 (NCT03228667, incorporated by reference herein).

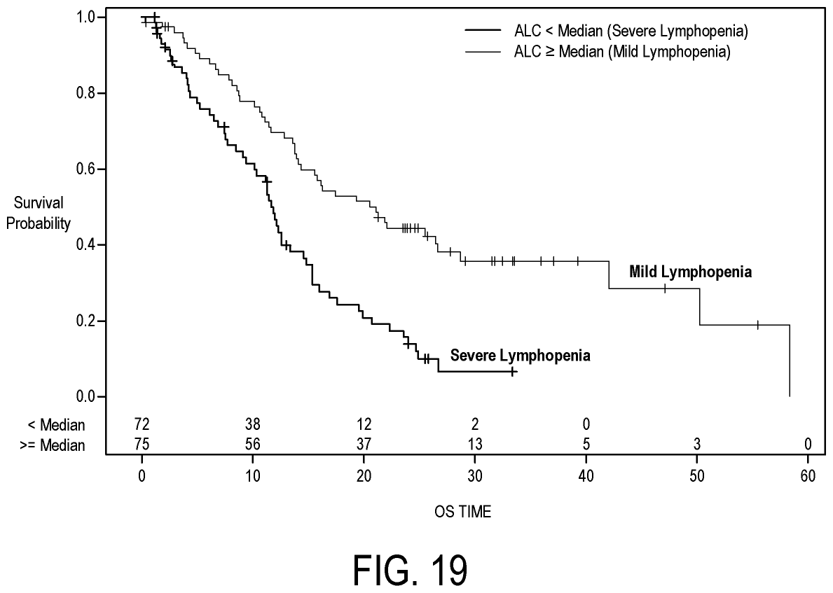

The mOS results across these multiple tumor types ranged from 12.6 to 19.3 months and the impact of median baseline ALC in effecting the duration of mOS was studied to determine the impact of lymphopenia. Baseline ALC of All Cohorts: Consistent with the findings of highly significant prolonged OS when the lymphocyte count is ≥the median baseline level of 1,200 lymphocytes in NSCLC as shown above, when the same analysis performed in all cohorts of multiple tumors (N=147), the highly significant finding is noted in prolonging OS when the baseline ALC exceeds the median of 1,200 lymphocytes per microliter (p-value<0.0001; hazard ratio: 0.43). Table 4 shows all cohorts (N=147) across multiple tumor types: overall survival by baseline absolute lymphocyte count (ALC) median, and FIG. 19 depicts a Kaplan Meier plot for all cohorts (N=147) across multiple tumor types: Overall Survival is plotted against baseline absolute lymphocyte count (ALC) median. Such finding is particularly notable as overall survival was substantially increased in patients with CPI failure. With the reversal or alleviation of lymphopenia, it is therefore contemplated that CPI treatment can once more engage in a physiologically meaningful manner.

TABLE 4

| | Baseline ALC < | Baseline ALC ≥ | | |
|---|---|---|---|---|
| | Median (1,200) | Median (1,200) | | Hazard Ratio |
| Variable | N = 72 | N = 75 | P-Value | (95% CI) |
| Number of Deaths | 56 (78%) | 47 (63%) | | |
| Median Survival (months) | 11.7 | 21.1 | <0.0001 | 0.43 (0.28, 0.64) |
| 95% CI for the Median Survival | 9.1, 14.6 | 14.1, 26.6 | | |

### Lynch Syndrome (AdV/N-803)

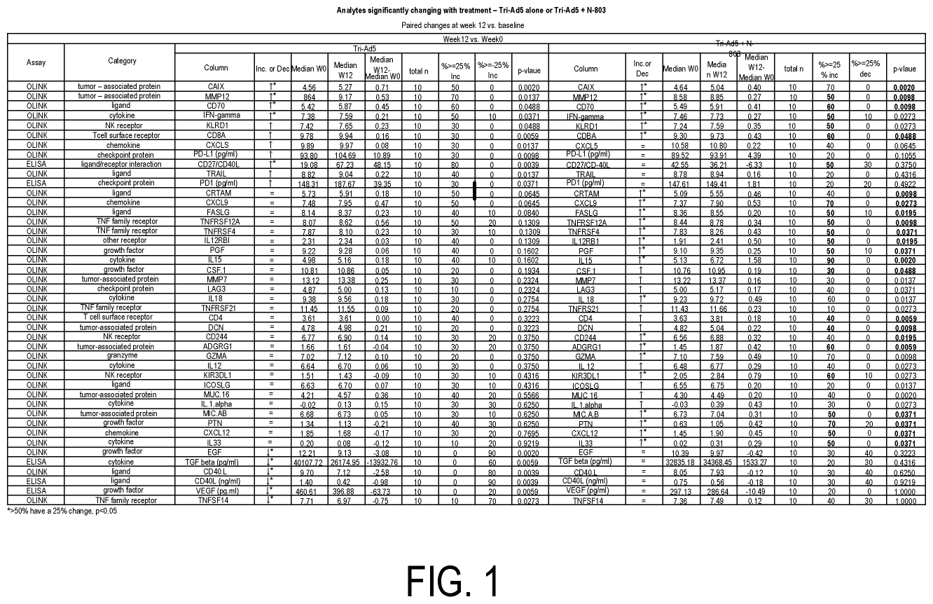

In view of the above data and other considerations, combination therapy is also deemed to be effective for subjects with idiopathic or virally induced lymphopenia. In one preliminary study, a group of subjects with Lynch syndrome were treated with Tri-Ad5 vaccine targeting CEA/MUC1/Brachyury (recombinant adenovirus with recombinant nucleic acid encoding CEA/MUC1/Brachyury) alone, while another group of subjects received the same treatment in combination with N-803. The subjects were followed over 12 weeks with a four-week interim analysis, and various parameters and analytes were investigated as exemplarily shown in the Table of FIG. 1.

Moreover, there were both treatment-specific and unique changes in serum analytes from baseline to 4 weeks. Patients given Tri-Ad5 alone and Tri-Ad5 plus N-803 each had increases in some T cell and NK cell related analytes, although the increase in NK cell analytes and T and NK cell-related pathways was more pronounced when patients received N-803 plus Tri-Ad5. Patients given Tri-Ad5 alone had more decrease in immunosuppressive analytes compared to patients given N-803 plus Tri-Ad5.

In a direct comparison of treatment arms, patients given Tri-Ad5 plus N-803 had higher absolute levels of T cell-recruiting chemokine CXCL11 and lower levels of PD1 than patients receiving Tri-Ad5 alone, and less of an increase in CD27/CD40L. When comparing to treatment arms, patients receiving N-803 plus Tri-Ad5 had greater increases in multiple immune related pathways and greater decreases in immune suppressive pathways compared to patients receiving Tri-Ad5 alone. Overall, at four weeks, serum analyses indicated that while Tri-Ad5 appears to enhance T cell activity, the addition of N-803 further enhances T cell activity along with increases in NK cell activity. However, less reduction of several immunosuppressive analytes was seen with Tri-Ad5 plus N-803 compared to Tri-Ad5 alone.

Summary of serum findings of 12 weeks: There were both treatment-specific and unique changes in serum analytes from baseline to 12 weeks, with patients given N-803 having a greater number of increased analytes. Patients given Tri-Ad5 alone and Tri-Ad5 plus N-803 each had increases in some T cell and NK cell related analytes, although the increase in NK cell analytes and T and NK cell-related pathways was more pronounced when patients received N-803 plus Tri-Ad5. Patients given Tri-Ad5 alone had decreases in immunosuppressive serum analytes, compared to no change when patients received Tri-Ad5 plus N-803.

In a direct comparison of treatment arms, patients given Tri-Ad5 plus N-803 had higher absolute levels of NK-cell receptor KIR3DL1, immunocytokine IL-15, and granzymes A and B, along with a greater increase in IL-15 and less of a decrease in VEGF and immunosuppressive CD40L, than patients receiving Tri-Ad5 alone. When comparing treatment arms, patients receiving N-803 plus Tri-Ad5 had greater increases in both T cell and NK cell-related pathways than patients receiving Tri-Ad5 alone. Overall, serum analyses indicated that while Tri-Ad5 appears to enhance T cell activity, the addition of N-803 furthers this activation, while also enhancing NK cell activity. However, less reduction of several immunosuppressive analytes was seen with Tri-Ad5 plus N-803 compared to Tri-Ad5 alone.

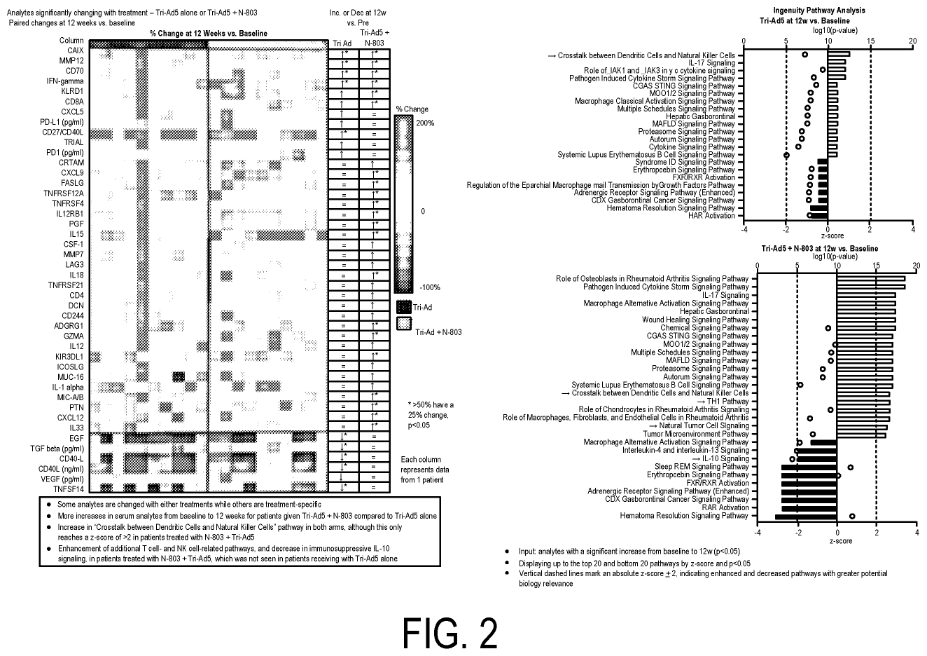

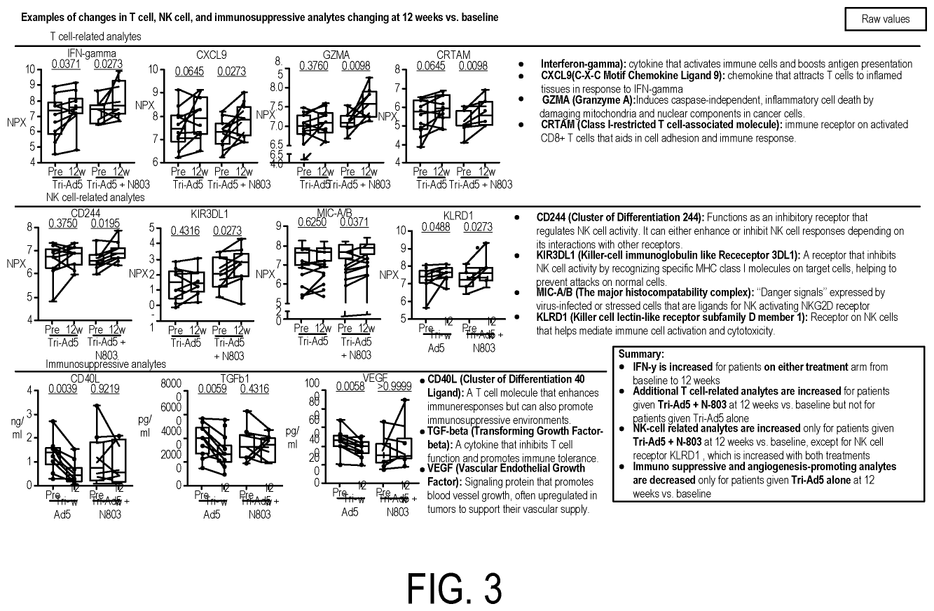

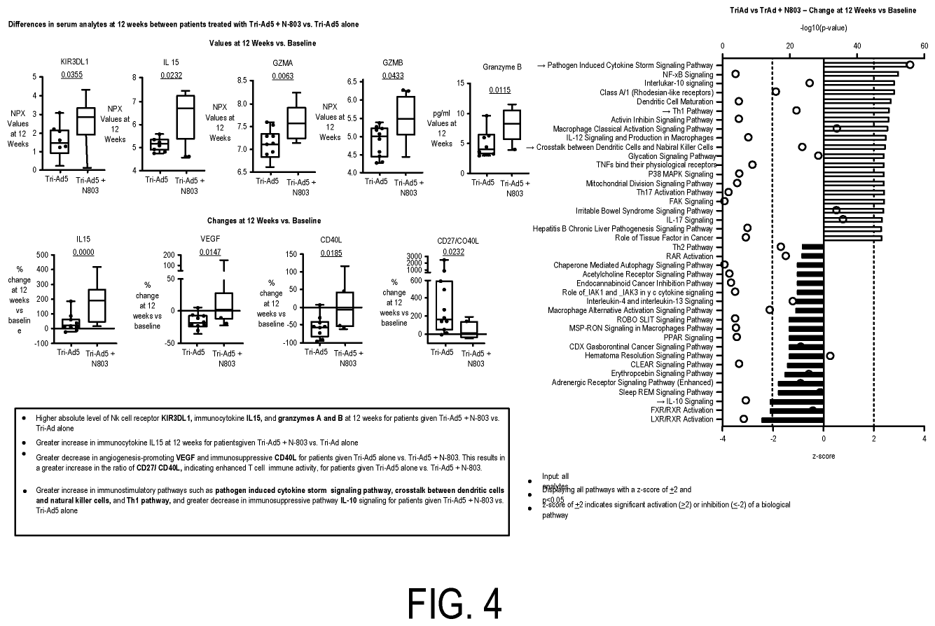

Summary of serum findings at 12 compared to 4 weeks: Greater differences in immune activation between patients receiving N-803 plus Tri-Ad5 compared to patients receiving Tri-Ad5 alone are seen after multiple cycles of treatment (12 weeks) as compared to after a single cycle of treatment (at 4 weeks). At 12 weeks, there are greater increases in immune pathways indicating enhanced T cell and NK cell activity with the addition of N-803 compared to Tri-Ad5 alone, including “pathogen induced cytokine storm signaling pathway”, “crosstalk between dendritic cells and natural killer cells”, and “Th1 pathway”, which was not seen at 4 weeks. An exemplary summary of results from this study is shown in FIG. 2, FIG. 3, and FIG. 4.

### Tri-AdV/Yeast N-803—Mouse

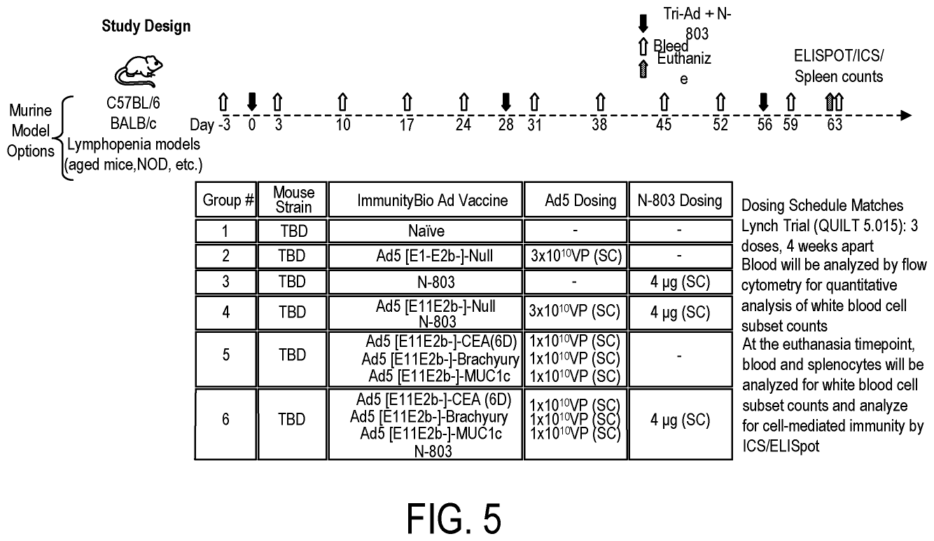

Additional efficacy and synergistic effects of TriAd5 plus N-803 treatment may be further ascertained in corresponding animal studies, and FIG. 5 provides an exemplary overview of such experiment. Here, lymphocytopenic mice (e.g., aged or non-obese diabetic) will be subjected to s.c. injection of a mixture of recombinant Ad5 viruses, where a first fraction of the Ad5 viruses comprise a recombinant nucleic encoding CEA, where a second fraction of the Ad5 viruses comprise a recombinant nucleic encoding Brachyury, and where a third fraction of the Ad5 viruses comprise a recombinant nucleic encoding MUC-1c. This group will also receive an s.c. injection of N-803. As a control, mice will also be subjected to the same treatment, but without N-803 administration. As further controls, one group will be injected with Ad5 null vector (i.e., no recombinant cancer associated antigen) with N-803 administration, while another group will be injected with N-803 alone. Finally, one control group will remain treatment naïve.

As will be appreciated, the vaccine need not be limited to an adenoviral vaccine composition but may use a recombinant yeast vaccine in which the yeast cells express CEA/MUC1/Brachyury. Therefore, a Tri-Ad/Yeast plus N-803 lymphopenia study in C57BL/6 mice is contemplated where the dosing schedule used in this study matches that of the Lynch Trial (QUILT 5.015), consisting of three doses administered at four-week intervals.

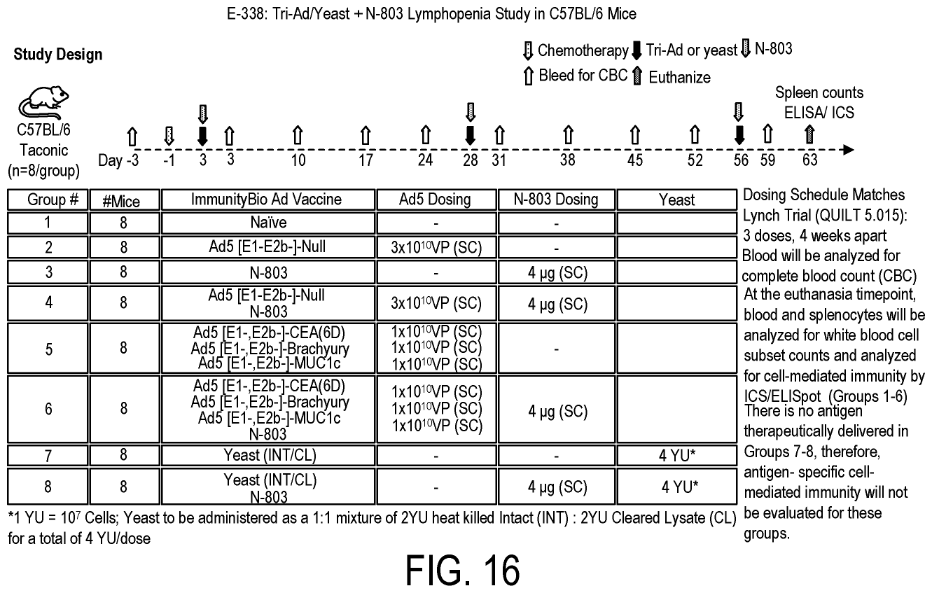

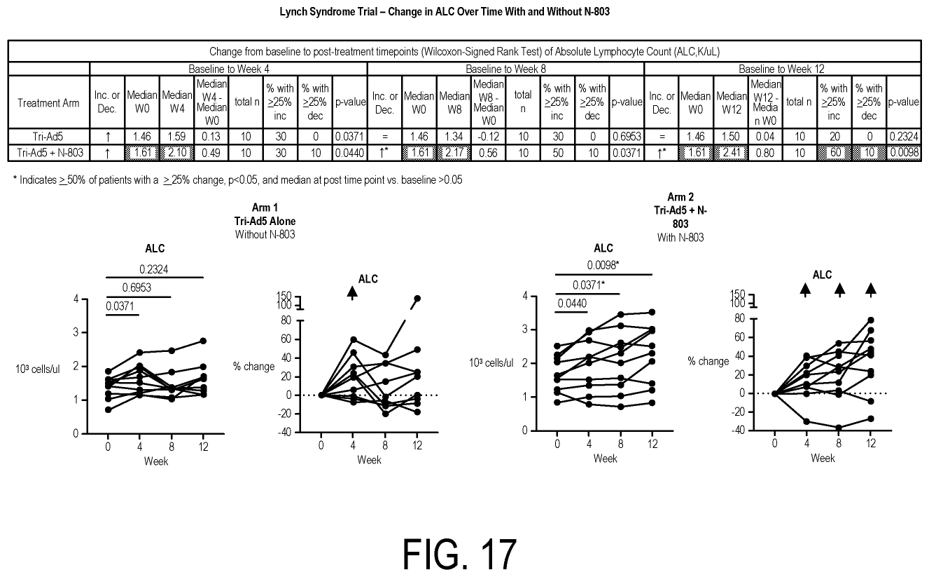

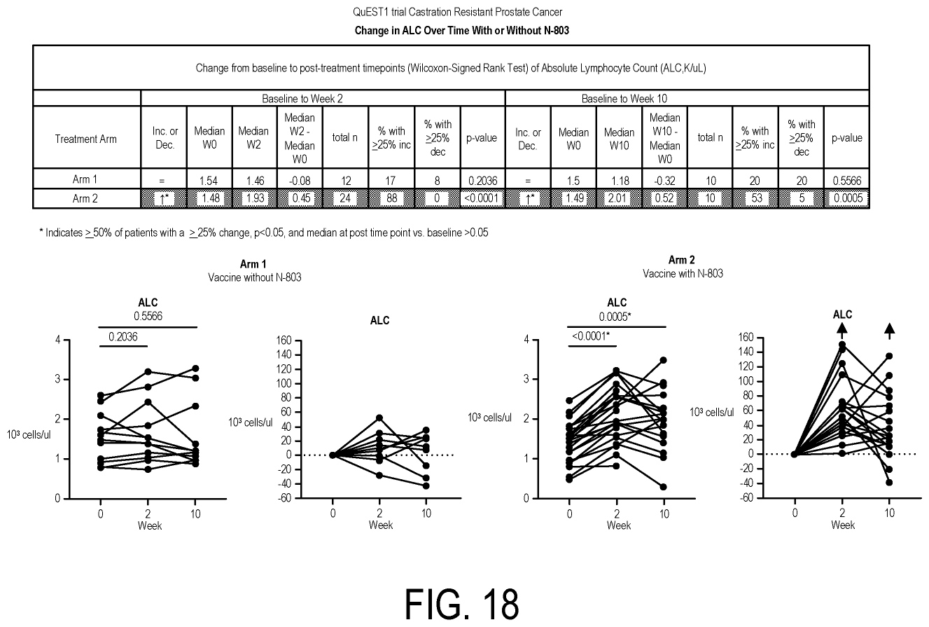

Throughout the study, blood samples will be collected and analyzed for complete blood count (CBC). At the designated euthanasia timepoint, additional analyses are performed on both blood and splenocyte samples to assess white blood cell subset counts. Furthermore, cell-mediated immunity is evaluated using intracellular cytokine staining (ICS) and ELISpot assays in Groups 1 through 6. Notably, Groups 7 and 8 do not receive any therapeutically delivered antigen; therefore, antigen-specific cell-mediated immune responses is not assessed in these groups. This is schematically illustrated in FIG. 16.

### Monocytopenia

In still further studies, the inventors also investigated whether N-803 administration could have a therapeutic effect on monocytopenia (characterized as reduction in blood monocyte count to <200/mcL). In this context, it should be recognized that monocytopenia is not a subset of lymphopenia. Rather, these conditions are distinct and separate that may or may not occur together. While monocytopenia is characterized by a lower-than-normal level of monocytes (white blood cells of the innate immune response that act as ‘first responders’ to pathogens as part), lymphopenia is characterized as a lower-than-normal level of lymphocytes (white blood cells of the adaptive immune response and natural killer cells).

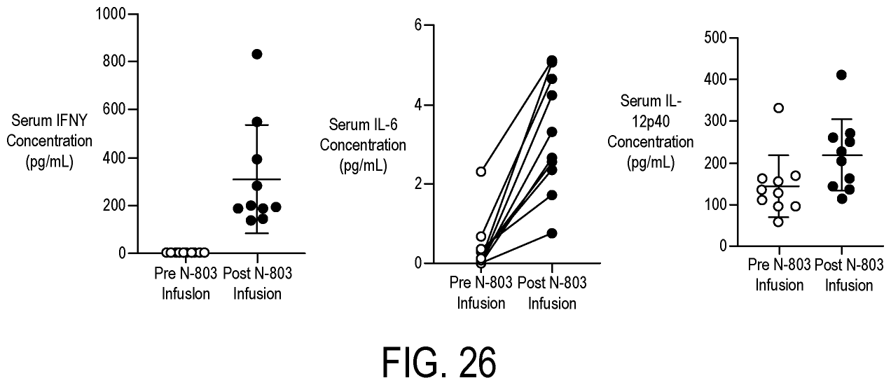

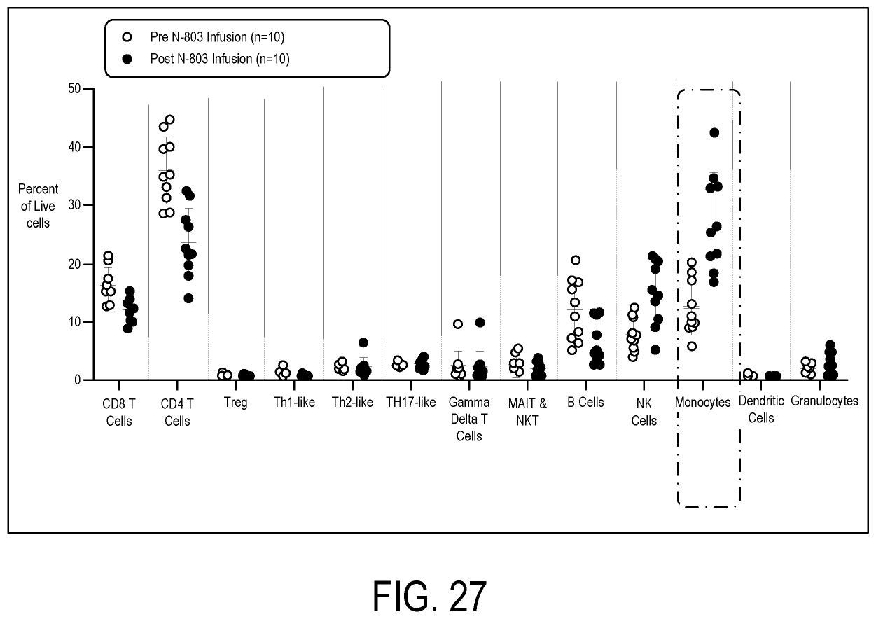

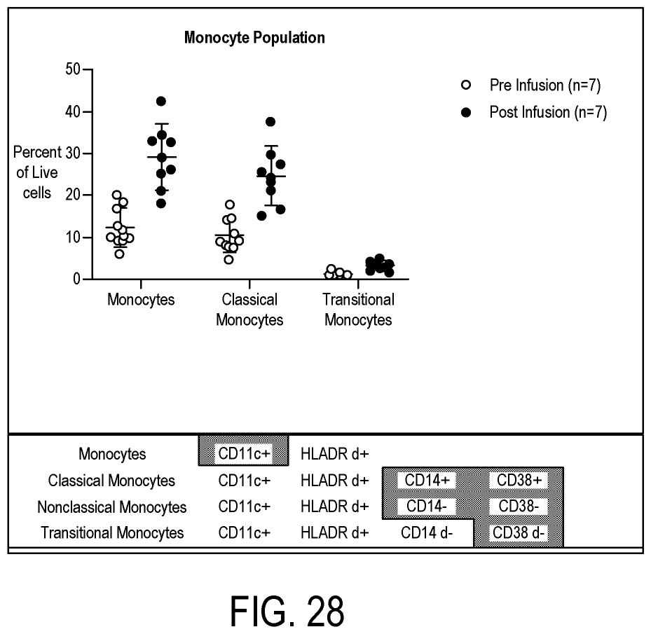

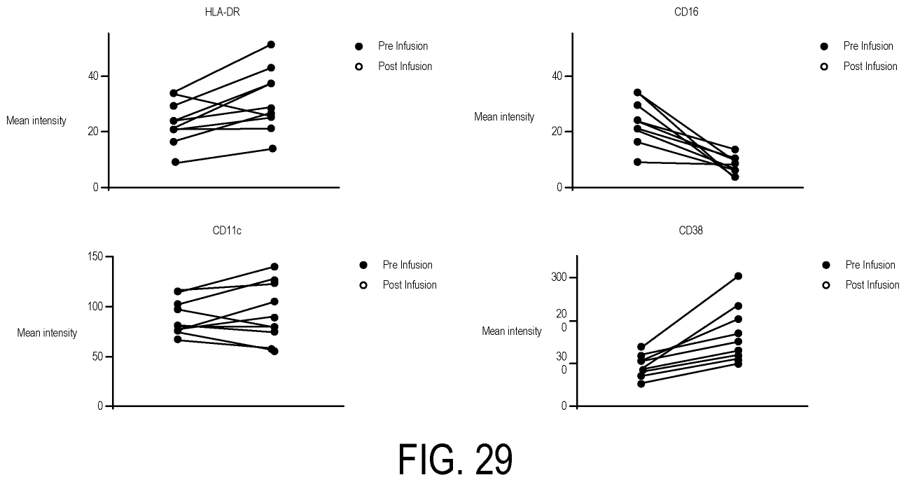

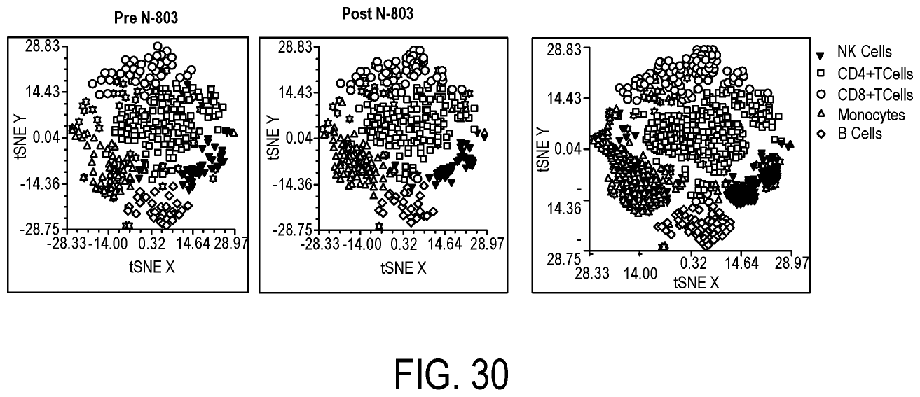

To that end, the inventors investigated blood count data from QUILT-105 (NCT06040918, incorporated by reference herein) is a single center study evaluating the effects of N-803 stimulation of the expansion, enrichment and proliferation ofNatural Killer cells from Apheresis Product. Ten healthy subjects were enrolled for the study and were scheduled for the first apheresis collection on Day 1. All subjects received N-803 at a fixed dose of 1 mg by subcutaneous (SC) injection 4 to 5 days prior to the second apheresis collection. The second apheresis collection occurred between Day 12 and Day 29. Remarkably, N-803 not only demonstrated an increase in NK cells, but also unexpectedly showed a substantial stimulatory effect on monocytes and selected cytokines. An exemplary cytokine analysis post N-803 administration for IFN-gamma, IL-6, and IL-12p40 is shown in FIG. 26. The increase in monocytes post N-803 administration is depicted in FIG. 27, while FIG. 28 shows cell subtype distribution of the monocyte population. FIG. 29 depicts change in immune cell composition pre/post N-803 as measured by representative markers, while FIG. 30 show T-SNE analysis of pre/post N-803 administration.

### Aspects

The present disclosure will be better understood upon reading the following numbered aspects which should not be confused with the claims. In some instances, each of the aspects described below can be combined with other aspects, including combined with other aspects described elsewhere in the disclosure or other aspects from the examples below, without departing from the spirit of the disclosure.

1. A method of treatment for a patient with lymphopenia, the method comprising: administering to the patient an effective amount of an IL-15N72D:IL-15RαSu/Fc complex (N-803) formulated for administration to a subject, wherein said N-803 comprises a dimeric IL-15RαSu/Fc and two IL-15N72D molecules.

2. The method of aspect 1, wherein the patient has previously been treated with chemotherapy and/or radiotherapy.

3. The method of aspect 2, wherein the chemotherapy comprises at least one of a taxane, a platinum-based agent, cyclophosphamide, fludarabine, an m-TOR inhibitor, a tyrosine kinase inhibitor, or methotrexate.

4. The method of aspect 1, wherein the patient has cancer.

5. The method of aspect 1, wherein the patient has non-small cell lung cancer (NSCLC).

6. The method of aspect 1, wherein the patient has an infectious disease.

7. A method of treatment for a patient with lymphopenia, the method comprising: administering to the patient an effective amount of 1) an adenoviral vector comprising at least one target antigen, and 2) an IL-15N72D:IL-15RαSu/Fc complex (N-803) formulated for administration to a subject, wherein said N-803 comprises a dimeric IL-15RαSu/Fc and two IL-15N72D molecules.

8. The method of aspect 7, wherein dendritic cells are activated.

9. The method of aspect 7, wherein granzyme and perforin are upregulated.

10. The method of aspect 7, wherein Interleukin-15 (IL-15) is upregulated.

11. The method of aspect 7, wherein Interleukin-10 (IL-10) signaling is decreased.

12. The method of aspect 7, wherein interferon gamma (IFN7) signaling is upregulated.

13. The method of aspect 7, wherein crosstalk between dendritic cells and natural killer (NK) cells is increased.

14. The method of aspect 7, wherein crosstalk between dendritic cells and T cells is increased.

15. The method of aspect 7, wherein Th1 signaling is upregulated.

16. The method of aspect 7, wherein NK cells are activated.

17. The method of aspect 7, wherein CD4 T cells, CD8 T cells, and memory T cells are activated.

18. The method of aspect 7, wherein T cell activation peaks at 12 weeks.

19. The method of aspect 7, wherein the adenoviral vector comprises a replication defective adenoviral vector subtype 5 (Ad5) comprising a deletion in the E1 region and a deletion in the E2b region.

20. The method of aspect 19, wherein the Ad5 vector comprises a nucleic acid encoding CEA.

21. The method of aspect 19, wherein the Ad5 vector comprises a nucleic acid encoding Brachyury.

22. The method of aspect 19, wherein the Ad5 vector comprises a nucleic acid encoding MUC-1.

23. The method of aspect 19, wherein the Ad5 vector comprises a Tri-Ad5 vaccine, wherein the Tri-Ad5 vaccine comprises an Ad5 vector comprising a nucleic acid encoding CEA, an Ad5 vector comprising a nucleic acid encoding Brachyury, and an Ad5 vector comprising a nucleic acid encoding MUC-1.

24. The method of aspect 7, wherein the patient has been previously treated with radiotherapy, chemotherapy, and/or immunotherapy.

25. The method of aspect 24, wherein the immunotherapy comprises an immune checkpoint inhibitor.

26. The method of aspect 25, wherein the immune checkpoint inhibitor comprises an anti-PD-1, anti-PD-L1, or anti-CTLA4 antibody.

27. The method of aspect 25, wherein the immune checkpoint inhibitor comprises avelumab, nivolumab, atezolizumab, durvalumab, tremelimumab, or pembrolizumab.

28. A method of treatment for a patient with immune checkpoint inhibitor induced lymphopenia, the method comprising: administering to the patient who has been previously treated with a first immune checkpoint inhibitor an effective amount an IL-15N72D:IL-15RαSu/Fc complex (N-803) formulated for administration to a subject; wherein said N-803 comprises a dimeric IL-15RαSu/Fc and two IL-15N72D molecules.

29. The method of aspect 28, wherein the patient has cancer.

30. The method of aspect 28, wherein the patient has non-small cell lung cancer (NSCLC).

31. The method of aspect 28, wherein the first immune checkpoint inhibitor comprises an anti-PD-1, anti-PD-L1, or anti-CTLA4 antibody.

32. The method of aspect 28, wherein the first checkpoint inhibitor comprises avelumab, nivolumab, atezolizumab, durvalumab, tremelimumab, or pembrolizumab.

33. The method of aspect 28, wherein the patient is further administered a second immune checkpoint inhibitor.

34. The method of aspect 33, wherein the N-803 and the second immune checkpoint inhibitor are administered simultaneously.

35. The method of aspect 33, wherein the N-803 and the second immune checkpoint inhibitor are administered sequentially.

36. The method of aspect 33, wherein the second immune checkpoint inhibitor comprises an anti-PD-1, anti-PD-L1, or anti-CTLA4 antibody.

37. The method of aspect 33, wherein the second checkpoint inhibitor comprises avelumab, nivolumab, atezolizumab, durvalumab, tremelimumab, or pembrolizumab.

38. A method of treatment for a patient with lymphopenia, the method comprising: administering to the patient an effective amount of 1) an adenoviral vector comprising at least one target antigen, and

2) an IL-15:IL-15Ra/IL-7 complex (HCW-9210) formulated for administration to a subject, wherein said HCW-9210 comprises a multi-chain chimeric polypeptide comprising: (a) a first chimeric polypeptide comprising IL-7, Tissue Factor, and IL-15; and (b) a second chimeric polypeptide comprising IL-7 and IL-15Ra, wherein the first chimeric polypeptide and the second chimeric polypeptide associate through the binding of the IL-15 and the IL-15Ra.

39. The method of aspect 38, wherein the adenoviral vector comprises a replication defective adenoviral vector subtype 5 (Ad5) comprising a deletion in the E1 region and a deletion in the E2b region.

40. The method of aspect 39, wherein the Ad5 vector comprises a nucleic acid encoding CEA.

41. The method of aspect 39, wherein the Ad5 vector comprises a nucleic acid encoding Brachyury.

42. The method of aspect 39, wherein the Ad5 vector comprises a nucleic acid encoding MUC-1.

43. The method of aspect 39, wherein the Ad5 vector comprises a Tri-Ad5 vaccine, wherein the Tri-Ad5 vaccine comprises an Ad5 vector comprising a nucleic acid encoding CEA, an Ad5 vector comprising a nucleic acid encoding Brachyury, and an Ad5 vector comprising a nucleic acid encoding MUC-1.

44. The method of aspect 38, wherein the patient has been previously treated with radiotherapy, chemotherapy, and/or immunotherapy.

45. The method of aspect 44, wherein the immunotherapy comprises an immune checkpoint inhibitor.

46. The method of aspect 45, wherein the immune checkpoint inhibitor comprises an anti-PD-1, anti-PD-L1, or anti-CTLA4 antibody.

47. The method of aspect 45, wherein the immune checkpoint inhibitor comprises avelumab, nivolumab, atezolizumab, durvalumab, tremelimumab, or pembrolizumab.

48. A method of treatment for a patient with lymphopenia, the method comprising: administering to the patient an effective amount of 1) an adenoviral vector comprising at least one target antigen, 2) an IL-15N72D:IL-15RαSu/Fc complex (N-803) formulated for administration to a subject, and 3) a CAR Natural Killer (CAR NK) cell, wherein said N-803 comprises a dimeric IL-15RαSu/Fc and two IL-15N72D molecules.

49. The method of aspect 48, wherein the CAR NK cell comprises a natural killer cell line.

50. The method of aspect 49, wherein the natural killer cell line is an NK-92 cell.

51. The method of aspect 48, wherein the CAR NK cell is a primary human NK cell.

52. The method of aspect 51, wherein the primary human NK cell is a memory like cytokine enhanced NK (m-ceNK) cell, or a cytokine induced memory-like (CIML) NK cell.

53. The method of aspect 48, wherein the CAR comprises an extracellular binding domain with specificity for PD-L1.

54. The method of aspect 48, wherein the CAR comprises an extracellular binding domain with specificity for EGFR or HER2.

55. A method of treatment for a patient with lymphopenia, the method comprising: administering to the patient an effective amount of 1) IL-7 or stabilized derivates thereof, and 2) an IL-15N72D:IL-15RαSu/Fc complex (N-803) formulated for administration to a subject, wherein said N-803 comprises a dimeric IL-15RαSu/Fc and two IL-15N72D molecules.

56. The method of aspect 55, wherein the patient has previously been treated with chemotherapy and/or radiotherapy.

57. The method of aspect 56, wherein the chemotherapy comprises at least one of a taxane, a platinum-based agent, cyclophosphamide, fludarabine, an m-TOR inhibitor, a tyrosine kinase inhibitor, or methotrexate

58. The method of aspect 55, wherein the patient has cancer.

59. The method of aspect 55, wherein the patient has non-small cell lung cancer (NSCLC).

60. The method of aspect 55, wherein the patient has an infectious disease.

61. A method of treating lymphopenia and/or tumor in a patient in need thereof, the method comprising: administering to the patient an effective amount of 2) an IL-15N72D:IL-15RαSu/Fc complex (N-803), and 3) a Natural Killer (NK) cell.

62. The method of aspect 61, wherein the NK cell comprises a CAR NK-92 cell.

63. The method of aspect 62, wherein the CAR comprises an extracellular binding domain with specificity for PD-L1.

64. The method of any one of aspects 61-63, wherein the method is for treatment of pancreatic cancer.

65. The method of aspect 61, wherein the NK cell is a cytokine enhanced NK (m-ceNK) cell.

66. The method of aspect 66, wherein the method is for treatment of advanced and metastatic solid tumors.

67. The method of any one of aspects 61-66, wherein the N-803 is administered to the patient via injection.

68. The method of aspect 67, wherein the injection is a subcutaneous injection.

69. The method of any one of aspects 61-68, wherein the NK cell is administered to the patient via infusion.

70. A method of providing a protective bioshield against cancer, the method comprising: administering to a patient in need thereof a combination of N-803 and at least one immunotherapy agent, wherein the method enhances the patient's defense against cancer.

71. The method of aspect 70, wherein the at least one immunotherapy agent is selected from the group consisting of: checkpoint inhibitors (CPI), NK cell therapies, m-ceNK cell therapies, t-haNK cell therapies, recombinant adenoviral vectors such as Ad5 expressing tumor-associated antigens, yeast-based vaccines or vectors, HDAC inhibitors, and cytokines such as interleukin-7 (IL-7), interleukin-21 (IL-21).

72. The method of any one of aspects 70-71, wherein the N-803 is administered to the patient via injection.

73. The method of aspect 72, wherein the injection is a subcutaneous injection.

74. A method of treatment for a patient with lymphopenia, the method comprising: administering to the patient a) an effective amount of an IL-15N72D:IL-15RαSu/Fc complex (N-803) formulated for administration to the patient, and b) an effective amount of granulocyte colony stimulating factor (G-CSF) formulated for administration to the patient.

75. The method of aspect 74, wherein the patient has been or will be treated with chemotherapy.

76. The method of aspect 75, wherein the chemotherapy comprises at least one of a taxane, a platinum-based agent, cyclophosphamide, fludarabine, an m-TOR inhibitor, a tyrosine kinase inhibitor, doxorubicin, etoposide, ifosfamide, vinblastine, or methotrexate.

77. The method of aspect 75, wherein the G-CSF is administered before chemotherapy.

78. The method of aspect 75, wherein the G-CSF is administered after chemotherapy.

79. The method of aspect 75, wherein the G-CSF is administered before and after chemotherapy.

80. The method of aspect 74, wherein the patient has been or will be treated with radiotherapy (RT).

81. The method of aspect 80, wherein the N-803 is administered before RT.

82. The method of aspect 80, wherein the N-803 is administered after RT.

83. The method of aspect 80, wherein the N-803 is administered before and after RT.

84. The method of aspect 74, wherein the patient has cancer.

85. The method of aspect 74, wherein the N-803 is administered at 10-50 μg/kg.

86. The method of aspect 74, wherein the N-803 is administered at 15 μg/kg.

87. The method of aspect 74, wherein the G-CSF is administered at 1-100 μg/kg.

88. A method of providing a protective bioshield against cancer in a patient in need thereof, wherein the bioshield comprises an induced cytotoxic (CD8+) and helper (CD4+) T cell-mediated immune response to the cancer, the method comprising: administering to the patient a combination of effective amounts of N-803 and G-CSF.

89. The method of aspect 88, further comprising administration of at least one immunotherapy agent selected from the group consisting of: checkpoint inhibitors (CPI), NK cell therapies, m-ceNK cell therapies, t-haNK cell therapies, recombinant adenoviral vectors such as Ad5 expressing tumor-associated antigens, yeast-based vaccines or vectors, HDAC inhibitors, and cytokines such as interleukin-7 (IL-7), interleukin-21 (IL-21).

90. The method of aspect 88, wherein the N-803 is administered to the patient via injection.

91. The method of aspect 90, wherein the injection is a subcutaneous or IV injection.

92. The method of aspect 88, wherein the G-CSF is administered to the patient via injection.

93. The method of aspect 92, wherein the injection is a subcutaneous or IV injection.

94. A composition for use in the treatment for a patient with lymphopenia, the composition comprising: a) an effective amount of an IL-15N72D:IL-15RαSu/Fc complex (N-803) formulated for administration to the patient, and b) an effective amount of granulocyte colony stimulating factor (G-CSF) formulated for administration to the patient.

95. The composition of aspect 94, wherein the N-803 and G-CSF are formulated together for simultaneous administration to the patient.

96. The composition of aspect 94, wherein the N-803 and G-CSF are formulated separately for simultaneous administration to the patient.

97. The composition of aspect 94, wherein the N-803 and G-CSF are formulated separately for sequential administration to the patient.

98. A kit for use in the treatment for a patient with lymphopenia, the kit comprising: a) a first vial comprising an effective amount of an IL-15N72D:IL-15RαSu/Fc complex (N-803) formulated for administration to the patient, b) a second vial comprising an effective amount of granulocyte colony stimulating factor (G-CSF) formulated for administration to the patient, c) at least one bag containing a sterile saline solution; and d) directions for use thereof.

99. The kit of aspect 98, wherein the N-803 and G-CSF are formulated together for simultaneous administration to the patient.

100. The kit of aspect 98, wherein the N-803 and G-CSF are formulated separately for simultaneous administration to the patient.

101. The kit of aspect 98, wherein the N-803 and G-CSF are formulated separately for sequential administration to the patient.

102. A method of treatment for a patient with lymphopenia, the method comprising: a. administering to the patient an effective amount of 1) IL-7 or a stabilized derivate thereof; 2) an IL-15N72D:IL-15RαSu/Fc complex (N-803) formulated for administration to a subject, wherein said N-803 comprises a dimeric IL-15RαSu/Fc and two IL-15N72D molecules; and 3) G-CSF.

103. The method of aspect 102, wherein the patient has previously been treated with chemotherapy and/or radiotherapy.

104. The method of aspect 103, wherein the chemotherapy comprises at least one of a taxane, a platinum-based agent, cyclophosphamide, fludarabine, an m-TOR inhibitor, a tyrosine kinase inhibitor, doxorubicin, etoposide, ifosfamide, vinblastine, or methotrexate.

105. The method of aspect 102, wherein the patient has cancer.

106. The method of aspect 102, wherein the patient is concurrently being treated with chemotherapy and/or radiotherapy.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID No:1-4 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:- (i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants. Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance. Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, suitable calculations of the percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:- Sequence Identity=(N/T)*100. Alternative methods for identifying similar sequences will be known to those skilled in the art. For example, a substantially similar nucleotide sequence will be encoded by a sequence which hybridizes to DNA sequences or their complements under stringent conditions. By stringent conditions, we mean the nucleotide hybridizes to filter-bound DNA or RNA in 3× sodium chloride/sodium citrate (SSC) at approximately 45° C. followed by at least one wash in 0.2× SSC/0.1% SDS at approximately 20-65° C.

Alternatively, a substantially similar polypeptide may differ by at least 1, but less than 5, 10, 20, 50 or 100 amino acids from the sequences shown in, for example, in the amino acid sequence that are included within SEQ ID Nos: 1-4. Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

In some embodiments, the numbers expressing quantities of ingredients, properties such as concentration, reaction conditions, and so forth, used to describe and claim certain embodiments of the invention are to be understood as being modified in some instances by the term “about.” As used herein, the terms “about” and “approximately”, when referring to a specified, measurable value (such as a parameter, an amount, a temporal duration, and the like), is meant to encompass the specified value and variations of and from the specified value, such as variations of +/−10% or less, alternatively +/−5% or less, alternatively +/−1% or less, alternatively +/−0.1% or less of and from the specified value, insofar as such variations are appropriate to perform in the disclosed embodiments. Thus, the value to which the modifier “about” or “approximately” refers is itself also specifically disclosed. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

What is claimed is:

1. A method of treatment of a cancer patient with lymphopenia, wherein the lymphopenia was induced by prior cancer treatment, the method comprising:
administering to the patient an effective amount of an JL-15N72D:IL-15RαSu/Fc complex (N-803) formulated for administration to a subject, wherein said N-803 comprises a dimeric IL-15RαSu/Fc and two IL-15N72D molecules; and
wherein the N-803 is administered over a period and at a schedule effective to increase absolute lymphocyte count (ALC) to thereby increase median overall survival.

2. The method of claim 1, wherein the prior cancer treatment is selected form the group consisting of checkpoint inhibitor treatment, radiation treatment, chemotherapy, and immune therapy.

3. The method of claim 1, wherein the N-803 is administered subcutaneously over at least four dosages.

4. The method of claim 1, wherein the ALC is increased to at least 1,000 cells/μL.

5. The method of claim 1, further comprising a step of co-administering at least one of a checkpoint inhibitor, a cancer vaccine, a cell-based product, and granulocyte colony stimulating factor (G-CSF) and a cytokine.

6. The method of claim 1, wherein the cell-based product comprises a memory like cytokine enhanced NK (m-ceNK) cell, a cytokine induced memory-like (CIML) NK cell, an autologous NK cell, an NK cell expressing a high-affinity CD16 receptor (haNK cell), or a targeted haNK cell expressing a chimeric antigen receptor that binds to a tumor epitope.

7. The method of claim 1, wherein the cancer patient has a solid tumor.

8. A method of increasing median overall survival in a cancer patient with lymphopenia, wherein the lymphopenia was induced by prior cancer treatment, the method comprising:
co-administering to the patient (a) a cancer treatment and (b) an effective amount of an IL-15N72D:IL-15RαSu/Fc complex (N-803) formulated for administration to a subject, wherein said N-803 comprises a dimeric IL-15RαSu/Fc and two IL-15N72D molecules.

9. The method of claim 8, wherein the cancer treatment is the prior cancer treatment.

10. The method of claim 8, wherein the effective amount is effective to increase the lymphocyte count of the patient to an ALC that is at least 1,000 cells/μL.

11. The method of claim 8, wherein the cancer treatment comprises administration of at least one of a checkpoint inhibitor, a cancer vaccine, a cell-based product, and granulocyte colony stimulating factor (G-CSF) and a cytokine.

12. The method of claim 8, wherein median overall survival is at least 4 months.

13. The method of claim 8, wherein the cancer patient has a solid tumor.

14. The method of claim 8, wherein the solid tumor is non-small-cell lung cancer (NSCLC), small-cell lung cancer (SCLC), urothelial cancer, head and neck squamous cell cancer, melanoma, renal cancer, gastric cancer, cervical cancer, hepatocellular carcinoma (HCC), colorectal cancer, or metastatic pancreatic cancer.

15. A method of increasing median overall survival in a relapsed cancer patient with lymphopenia, wherein the lymphopenia was induced by prior cancer treatment, the method comprising:
administering to the patient an effective amount of an IL-15N72D:IL-15RαSu/Fc complex (N-803) formulated for administration to a subject, wherein said N-803 comprises a dimeric IL-15RαSu/Fc and two IL-15N72D molecules; and
continuing the prior treatment while the patient receives the N-803.

16. The method of claim 15, wherein the patient has relapsed on checkpoint inhibitor treatment.

17. The method of claim 16, wherein the checkpoint inhibitor targets PD-1 or CTLA-4.

18. The method of claim 15, further comprising a step of co-administering a cancer vaccine and/or a cell-based therapy.

19. The method of claim 18, wherein the cancer vaccine comprises a recombinant adenovirus that comprises a nucleic acid that encodes at least one of CEA, MUC-1, and Brachyury, or a tumor and patient specific neoepitope.

20. The method of claim 18, wherein the cell-based therapy comprises a memory like cytokine enhanced NK (m-ceNK) cell, a cytokine induced memory-like (CIML) NK cell, an autologous NK cell, an NK cell expressing a high-affinity CD16 receptor (haNK cell), or a targeted haNK cell expressing a chimeric antigen receptor that binds to a tumor epitope.


SEQUENCE LISTING (4 sequences)

SEQ ID NO: 1 - protein, 485 aa, synthetic construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRENWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 217 aa, synthetic construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHITCPPPMSVEHADIWVKSYSLYSRERYICNSGFKRKAGTSSLTECVLNKATNVAHWTTPSLKCIR

SEQ ID NO: 3 - protein, 114 aa, synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 4 - protein, 297 aa, synthetic construct
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK